(19) United States
(12) Patent Application Publication
(10) Pub. No.: US 2010/0029693 A1
(43) Pub. Date: Feb. 4, 2010

(54) NOVEL PI3K DELTA INHIBITORS AND METHODS OF USE THEREOF

(76) Inventors: Jason DOUANGPANYA, Bothell, WA (US); Joel S. Hayflick, Seattle, WA (US); Kamal D. Puri, Lynnwood, WA (US)

Correspondence Address: MORRISON & FOERSTER LLP, 12531 HIGH BLUFF DRIVE, SUITE 100, SAN DIEGO, CA, 92130-2040, US

(21) Appl. No.: 12/538,748

(22) Filed: Aug. 10, 2009

Related U.S. Application Data
(63) Continuation of application No. 10/918,803, filed on Aug. 13, 2004.
(60) Provisional application No. 60/540,090, filed on Jan. 28, 2004.
(60) Provisional application No. 60/495,370, filed on Aug. 14, 2003.

(51) Int. Cl. A61K 31/52; C07D 473/00

(52) U.S. Cl. 514/263.21; 544/277

(57) ABSTRACT

The present invention relates generally to phosphoinositide 3-kinases (PI3Ks), and more particularly to specific, selective, improved PI3Kδ inhibitors and methods of inhibiting undesirable levels of PI3Kδ activity using these PI3Kδ inhibitors to treat disorders mediated by PI3Kδ.

### CROSS-REFERENCE TO RELATED APPLICATIONS

This application is a continuation of U.S. application Ser. No. 10/918,803 filed on Aug. 13, 2004, which claims the benefit under 35 U.S.C. §119(e) of U.S. provisional patent application Ser. Nos. 60/495,370 filed Aug. 14, 2003, and 60/540,090 filed Jan. 28, 2004, the entire disclosures of which are incorporated herein by reference.

### FIELD OF THE INVENTION

The invention relates generally to phosphoinositide 3-kinases (PI3Ks), and more particularly to methods of inhibiting undesirable immune responses without inhibiting desired immune responses.

### BACKGROUND OF THE INVENTION

Immune responses including but not limited to inflammatory responses may result from infection with pathogenic organisms and viruses, noninfectious means such as trauma or reperfusion following myocardial infarction or stroke, immune responses to foreign antigens, and autoimmune diseases. Inflammatory responses are notably associated with the influx of leukocytes and/or leukocyte chemotaxis. Leukocytes provide a first line of immune defense against many common microorganisms.

The recruitment of leukocytes into inflamed tissues is dependent upon a series of adhesive events that occur between these cells and the endothelial cells of the microvasculature [Springer, Cell 76:301-314 (1994); and, Butcher, et al., Science 272:60-66 (1996)]. Tissue injury initiates this adhesion process by locally releasing mediators of inflammation including but not limited to histamine, TNFα and IL-1 that rapidly convert the endothelial cell surface to a proadhesive state. The conversion of the endothelial cell surface to a proadhesive state includes the upregulation of P-selectin and E-selectin on the luminal surface of blood vessels. P-selectin and E-selectin subsequently interact with constitutively-expressed carbohydrate ligands on circulating leukocytes to promote rapid attachment and rolling of these cells in preparation for transendothelial migration.

Selectin-mediated adhesion is critical to transendothelial migration as it facilitates the engagement of secondary leukocyte adhesion receptors including but not limited to the β₂-integrins with intracellular adhesion molecules (ICAMs) expressed on the surface of inflamed vascular endothelium. Selectin-mediated adhesion requires leukocyte stimulation by locally-produced chemoattractants including but not limited to IL-8 and LTB₄, and subsequently results in integrin-mediated stabilization of interactions between these cells and the vasculature endothelial cells. Leukocytes eventually transmigrate across the endothelial cell barrier towards inflammatory foci in response to a bacterial and/or host-derived chemoattractant(s) [Luster, N. Engl. J. Med. 338:436-445 (1998)]. Failure to complete any of these steps will impede leukocyte accumulation in inflamed tissue, as evidenced by leukocyte adhesion deficiency syndromes I and II [Kishimoto, et al., Cell, 50:193-202 (1987); and, Etzioni, Pediatr. Res., 39:191-198 (1996)].

Class I phosphoinositide 3-kinases (PI 3-kinases; PI3Ks) are known to play a pivotal role in the ability of leukocytes to undergo chemotaxis as the lipid products they generate, including but not limited to phosphatidylinositol (3,4,5)-trisphosphate (PIP₃), are critical for promoting asymmetric F-actin synthesis, and thus leukocyte cell polarization [Wymann, et al., Immunol. Today, 21:260-264 (2000); Fruman, et al., Semin. Immunol., 14:7-18 (2002); Rickert, et al., Trends Cell Biol., 10:466-473 (2000); and, Weiner, et al., Nat. Cell Biol., 1:75-81 (1999)]. The function of class I PI3Ks, however, is not limited to directed migration, in that they are also required for phagocytosis and generation of oxygen radicals in response to chemoattractants including but not limited to fMLP [Arcaro, et al., Biochem. J., 298:517-520 (1994); Cadwallader, et al., J. Immunol., 169:3336-3344 (2002); Sasaki, et al., Science, 287:1040-1046 (2000); Ninomiya, et al., J. Biol. Chem., 269:22732-22737 (1994); and, Bharadwaj, et al., J. Immunol., 166:6735-6741 (2001)]. The ability of class I PI3Ks to regulate these processes in leukocytes relies on PIP₃ mediated recruitment of two lipid-binding protein kinases, phosphatidylinositol-dependent kinase 1 (PDK1) and protein kinase B/Akt, both of which can interact with this PI-derivative via their pleckstrin homology domains. Association of these kinases with PIP3 at the plasma membrane brings them into close proximity, facilitating the phosphorylation and activation of Akt by PDK1 [Cantley, Science, 296:1655-1657 (2002)]. These proteins are, in turn, responsible for many of the downstream signaling events associated with PI3K activity.

Structurally, class I PI3Ks exist as heterodimeric complexes, consisting of a p110 catalytic subunit and a p55, p85, or p101 regulatory subunit. There are four p110 catalytic subunits, which are classified as p110α, p110β, p110γ, and p110δ [Wymann, et al., Biochim. Biophys. Acta., 1436:127-150 (1998); and, Vanhaesebroeck, et al, Trends Biochem. Sci., 22:267-272 (1997)]. Class I PI3Ks can be further divided into two subclasses (Ia and Ib) based on their mechanism of activation. The class Ia subgroup contains p110α, p110α, and p110δ, each of which associates with the p85 regulatory protein and is activated by receptor tyrosine kinases [Wymann, et al., Biochim. Biophys. Acta., 1436:127-150 (1998); Cumock, et al., Immunology, 105:125-136 (2002); and, Stein, et al., Mol. Med. Today, 6:347-357 (2000)]. By contrast, the class Ib subgroup consists solely of p110γ, which associates with the p101 regulatory subunit, and is stimulated by G protein βγ subunits in response to chemoattractants. Neutrophils express all four members of class I P13Ks.

Evidence supporting the class I PI3Ks involvement in neutrophil cell migration is found in the ability of non-selective class I PI3K inhibitors, such as LY294002 and wortmannin, to mitigate neutrophil chemotaxis. Moreover, chemoattractant-directed migration of neutrophils has been reduced in mice deficient for p110γ catalytic subunit expression [Sasaki, et al., Science, 287:1040-1046 (2000); Knall, et al., Proc. Natl. Acad. Sci. U.S.A., 94:3052-3057 (1997); Hannigan, et al., Proc. Natl. Acad. Sci. U.S.A., 99:3603-3608 (2002); and, Hirsch, et al., Science, 287:1049-1053 (2000)]. The phosphoinositide 3-kinase (PI3K) catalytic subunit p110δ is thought to play a role at sites of inflammation by contributing solely to chemoattractant-directed neutrophil migration.

PI3K inhibitors that are selective for PI3Kδ have been disclosed in U.S. patent Publication 2002/161014 A1. Recently, the effects of a class I small molecule inhibitor specific for the PI3Kδ catalytic subunit have been studied [Sadhu, et al., J. Immunol., 170:2647-2654 (2003)]. This small molecule inhibitor was shown to block up to 65% of fMLP-induced PIP₃ generation in neutrophils as well as directed-migration of these cells on surface-immobilized ICAM-1 in response to this microbial product. Thus, Sadhu, et al. demonstrated that the lipid kinase activity of PI3Kδ is required for neutrophil directional migration to fMLP (using an under-agarose assay system). PI3Kδ inhibition affected both the number of neutrophils that were able-to migrate towards this bacterial product and the distance they were able to migrate.

Leukocyte accumulation in inflamed tissues relies on their ability to form adhesive interactions with inflamed vascular endothelium in response to chemoattractant-guided migration. Previously, it was known that the phosphoinositide 3-kinase (PI3K) catalytic subunit p110δ is expressed in neutrophils. In fact, previous reports suggest that PI3Kδ expression is largely restricted to leukocytes. The prior art, thus, merely suggests that p110δ plays a role at sites of inflammation by contributing solely to chemoattractant-directed neutrophil migration. A role for class I PI3Ks in inhibiting undesirable immune responses without inhibiting desired immune responses has not been suggested or demonstrated.

### SUMMARY OF THE INVENTION

The invention provides methods which inhibit an endogenous immune response stimulated by at least one endogenous factor without substantially inhibiting an exogenous immune response stimulated by at least one exogenous factor. The invention also provides methods which inhibit an endogenous immune response stimulated by at least one endogenous factor without substantially inhibiting immune responsiveness. Accordingly, the methods of the invention advantageously permit treatment of conditions associated with an undesirable endogenous immune response stimulated by at least one endogenous factor without compromising the ability to fight infection.

According to one embodiment of the invention, a method of inhibiting an endogenous immune response stimulated by at least one endogenous factor without substantially inhibiting an exogenous immune response stimulated by at least one exogenous factor comprises administering an amount of a phosphoinositide 3-kinase delta (PI3Kδ) selective inhibitor effective to inhibit the immune response stimulated by the at least one endogenous-factor without substantially inhibiting the exogenous immune response stimulated by the at least one exogenous factor.

According to another embodiment of the invention, a method of inhibiting an endogenous immune response stimulated by at least one endogenous factor without substantially inhibiting immune responsiveness comprises administering an amount of a phosphoinositide 3-kinase delta (PI3Kδ), selective inhibitor effective to inhibit the immune response stimulated by the at least one endogenous factor without substantially inhibiting immune responsiveness.

### DETAILED DESCRIPTION

The methods of the invention advantageously permit treatment of conditions associated with an undesirable endogenous immune response stimulated by at least one endogenous factor without substantially inhibiting an exogenous immune response stimulated by at least one exogenous factor. Thus, the methods of the invention provide methods of treating such undesirable endogenous immune responses without substantially compromising immune responsiveness including but not limited to the ability to fight infection. Furthermore, the methods may be used to prophylactically, i.e., to prevent onset and/or recurrence of conditions and/or symptoms associated with an undesirable endogenous immune response stimulated by at least one endogenous factor.

The invention provides methods of inhibiting an-endogenous immune response stimulated by at least one endogenous factor without substantially inhibiting an exogenous immune response stimulated by at least one exogenous factor comprising administering an amount of a phosphoinositide 3-kinase delta (PI3Kδ) selective inhibitor effective to inhibit the immune response stimulated by the at least one endogenous factor without substantially inhibiting the exogenous immune response stimulated by the at least one exogenous factor.

The immune response may be an inflammatory response. The immune response may be a leukocyte response. More specifically, the immune response may include one or more of: directed leukocyte migration; leukocyte superoxide production; leukocyte degranulation including but not limited to neutrophil elastase exocytosis; and, leukocyte transmigration and/or leukocyte extravasation. Leukocytes can be selected from the group consisting of neutrophils, eosinophils, basophils, T-lymphocytes, B-lymphocytes, monocytes, macrophages, dendritic cells, Langerhans cells, and mast cells.

As used herein, an “endogenous factor” is defined as a product which is synthesized by host cells, e.g., cells of the individual being treated. Representative endogenous factors include but are not limited to tumor necrosis factor alpha (TNF-alpha), complement factor C3a, complement factor C5a, chemokine CXCL1, chemokine CXCL2, chemokine CXCL3, chemokine CXCL4, chemokine CXCL5, chemokine CXCL6, chemokine CXCL7, interleukin 1 alpha (IL-1 alpha), interleukin 1 beta (IL-1 beta), interleukin 3 (IL-3), interleukin 6 (IL-6), interleukin 7 (IL-7), interleukin 8 (IL-8), interleukin 10 (IL-10), interleukin 11 (IL-11), interleukin 12 (IL-12), interleukin (IL-15), interleukin 17 (IL-17), interleukin 18 (IL-18), prostaglandins, monocyte chemoattractant protein-1 (MCP-1), chemokine CCL5 (RANTES), macrophage inflammatory protein-1-alpha (MIP-1-alpha), stromal cell-derived factor-1 (SDF-1), eotaxins, granulocyte-macrophage colony-stimulating factor (GM-CSF), transforming growth factor beta (TGF-beta), gamma-interferon (IFN-gamma), leukotriene B₄ (LTB₄), leukotriene C₄ (LTC₄), leukotriene D₄ (LTD₄), leukotriene E₄ (LTE₄), lipoxins, platelet-activating factor (PAF), and lysophospholipids.

As used herein, the term “without substantially inhibiting” means that an increase in compound concentration of at least about 10-fold is required to inhibit half-maximal of the response stimulated by exogenous factor. Accordingly, in one embodiment according to the invention, the compound concentration for administration in the methods of the invention is less than about 1/10 of the concentration needed to inhibit half-maximal of the response stimulated by exogenous factor.

As used herein, an “exogenous factor” is defined as a product of microbial origin. An exogenous factor may be released directly by a microbe or may comprise components or fragments of microbes (e.g., bacteria, fungi, protozoans, algae, yeast, and viruses) produced in response to phagosome mediated degradation by host cells. Representative exogenous factors include but are not limited to formyl-methionyl-leucyl-phenylalanine (fMLP), lipopolysaccharides (LPS), dsRNA, unmethylated nucleotides where cytosine is linked to guanine (unmethylated nucleotides CpG nucleotides), mannose-rich glycans, lipoproteins, peptidoglycans, lipoteichoic acid, lipoarabinomannan, mannans and mannoproteins, zymosan, and phosphorylcholine. Although LPS itself is not an effective chemoattractant, it can trigger an inflammatory response by stimulating the synthesis of endogenous cytokines and chemoattractants, such as TNFα and LTB₄, that promote leukocyte attachment to inflamed microvessels and directed migration of these cells [Xing, et al., Am. J. Pathol., 143:1009-1015 (1993); and, Yamasawa, et al., Inflammation, 23:263-274 (1999)].

As used herein, the term “PI3Kδ selective inhibitor” generally refers to a compound that inhibits the activity of the PI3Kδ isozyme more effectively than other isozymes of the PI3K family. A PI3Kδ selective inhibitor compound is therefore more selective for PI3Kδ than conventional PI3K inhibitors such as wortmannin and LY294002, which are “nonselective PI3K inhibitors.”

As used herein, the term “amount effective” means a dosage sufficient to produce a desired or stated effect.

According to another embodiment of the invention, a method of inhibiting an endogenous immune response stimulated by at least one endogenous factor without substantially inhibiting immune responsiveness comprises administering an amount of a phosphoinositide 3-kinase delta (PI3Kδ) selective inhibitor effective to inhibit the immune response stimulated by the at least one endogenous-factor without substantially inhibiting immune responsiveness.

In this embodiment according to the invention, the term “without substantially inhibiting” means that host clearance of a microbial infection still occurs when a compound in accordance with the invention is administered. As used herein, the term “immune responsiveness” refers to—the ability to resolve an infection of microbial origin.

The disclosed methods may inhibit immune responses mediated by one or more components of the PI3K/Akt pathway. Moreover, the disclosed methods may inhibit immune responses stimulated by at least one endogenous factor without substantially inhibiting one or more components of the p38 mitogen-activated kinase (p38 MAPK) pathway. The disclosed methods also may not substantially inhibit the following enzymes: Rac GTPase, PI3Kγ, and phosphodiesterases, specifically PDE4.

The ability of the methods and compounds in accordance with the invention to-inhibit an endogenous immune response stimulated by endogenous factor without substantially inhibiting an exogenous immune response stimulated by exogenous factor suggests that inhibition of PI3Kδ may be of therapeutic benefit in treatment of various conditions, e.g., conditions characterized by an inflammatory response including but not limited to autoimmune diseases, allergic diseases, and arthritic diseases. Importantly, inhibition of PI3Kδ function does not appear to affect biological functions such as viability and fertility.

“Inflammatory response” as used herein is characterized by redness, heat, swelling and pain (i.e., inflammation) and typically involves tissue injury or destruction. An inflammatory response is usually a localized, protective response elicited by injury or destruction of tissues, which serves to destroy, dilute or wall off (sequester) both the injurious agent and the injured tissue. Inflammatory responses are notably associated with the influx of leukocytes and/or leukocyte (e.g., neutrophil) chemotaxis. Inflammatory responses may result from infection with pathogenic organisms and viruses, noninfectious means such as trauma or reperfusion following myocardial infarction or stroke, immune responses to foreign antigens, and autoimmune diseases. Inflammatory responses amenable to treatment with the methods and compounds according to the invention encompass conditions associated with reactions of the specific defense system as well as conditions associated with reactions of the non-specific defense system.

As used herein, the term “specific defense system” refers to the component of the immune system that reacts to the presence of specific antigens. Examples of conditions characterized by a response of the specific defense system that are amenable to treatment in accordance with the invention include autoimmune diseases and delayed type hypersensitivity responses mediated by T-cells, chronic inflammatory diseases, transplant rejection, e.g., kidney and bone marrow transplants, and graft versus host disease (GVHD).

The term “non-specific defense system” as used herein refers to the component of the immune system that is incapable of immunological memory (e.g., granulocytes such as neutrophils, eosinophils, and basophils, mast cells, monocytes, macrophages). Examples of conditions characterized, at least in part, by a response of the non-specific defense system and amenable to treatment in accordance with the invention include adult (acute) respiratory distress syndrome (ARDS); multiple organ injury syndromes; reperfusion injury; acute glomerulonephritis; reactive arthritis; dermatitis with acute inflammatory components; acute purulent meningitis or other central nervous system inflammatory disorders such as stroke; thermal injury; inflammatory bowel disease; granulocyte transfusion associated syndromes; and cytokine-induced toxicity.

The therapeutic methods of the invention include methods for the amelioration of conditions associated with inflammatory cell activation. “Inflammatory cell activation” refers to the induction by a stimulus (including but not limited to, cytokines, antigens or auto-antibodies) of a proliferative cellular response, the production of soluble mediators (including but not limited to cytokines, oxygen radicals, enzymes, prostanoids, or vasoactive amines), or cell surface expression of new or increased numbers of mediators (including but not limited to, major histocompatibility antigens or cell adhesion molecules) in inflammatory cells (including but not limited to monocytes, macrophages, T lymphocytes, B lymphocytes, granulocytes (polymorphonuclear leukocytes including neutrophils, basophils, and eosinophils) mast cells, dendritic cells, Langerhans cells, and endothelial cells). It will be appreciated by persons skilled in the art that the activation of one or a combination of these phenotypes in these cells can contribute to the initiation, perpetuation, or exacerbation of an inflammatory condition

“Autoimmune disease” as used herein refers to any group of disorders in which tissue injury is associated with humoral or cell-mediated responses to the body's own constituents. “Transplant rejection” as used herein refers-to any immune response directed against grafted tissue (including organs or cells (e.g., bone marrow), characterized by a loss of function of the grafted and surrounding tissues, pain, swelling, leukocytosis, and thrombocytopenia. “Allergic disease” as used herein refers to any symptoms, tissue damage, or loss of tissue function resulting from allergy. “Arthritic disease” as used herein refers to any disease that is characterized by inflammatory lesions of the joints attributable to a variety of etiologies. “Dermatitis” as used herein refers to any of a large family of diseases of the skin that are characterized by inflammation of the skin attributable to a variety of etiologies.

As previously indicated, the methods of the invention are contemplated for the treatment of various conditions and/or disease states without compromising the ability to fight infection caused by exogenous factor(s). An individual need not be afflicted by an infection or other disease state caused by one or more exogenous factors in order for treatment in accordance with the methods and compounds of the invention to be indicated.

Autoimmune conditions which may be treated using an inhibitor of the invention include but are not limited to connective tissue disease, multiple sclerosis, systemic lupus erythematosus, rheumatoid arthritis, autoimmune pulmonary inflammation, Guillain-Barré syndrome, autoimmune thyroiditis, insulin dependent diabetes mellitus, myasthenia, gravis, graft-versus-host disease and autoimmune inflammatory eye disease. The inhibitors of the invention-may also be useful in the treatment of allergic reactions and conditions including but not limited to anaphylaxis, serum sickness, drug reactions, food allergies, insect venom allergies, mastocytosis, allergic rhinitis, hypersensitivity pneumonitis, urticaria, angioedema, eczema, atopic dermatitis, allergic contact dermatitis, erythema multiforme, Stevens-Johnson syndrome, allergic conjunctivitis, atopic keratoconjunctivitis, venereal keratoconjunctivitis, giant papillary conjunctivitis, contact allergies including but not limited to asthma (particularly, allergic asthma), and other respiratory problems.

Thus, in various embodiments, the invention provides methods of treating various inflammatory conditions including but not limited to arthritic diseases such as rheumatoid arthritis (RA), osteoarthritis, gouty arthritis, spondylitis, and reactive arthritis; Behcet's syndrome; sepsis; septic shock; endotoxic shock; gram negative sepsis; gram positive sepsis; toxic shock syndrome; multiple organ injury syndrome secondary to septicemia, trauma, or hemorrhage; ophthalmic disorders including but not limited to allergic conjunctivitis, vernal conjunctivitis, uveitis, and thyroid-associated ophthalmopathy; eosinophilic granuloma; pulmonary or respiratory conditions including but not limited to asthma, chronic bronchitis, allergic rhinitis, adult respiratory distress syndrome (ARDS), severe acute respiratory syndrome (SARS), chronic pulmonary inflammatory diseases (e.g., chronic obstructive pulmonary disease), silicosis, pulmonary sarcoidosis, pleurisy, alveolitis, vasculitis, pneumonia, bronchiectasis, hereditary emphysema, and pulmonary oxygen toxicity; ischemic-reperfusion injury, e.g., of the myocardium, brain, or extremities; fibrosis including but not limited to cystic fibrosis; keloid formation or scar tissue formation; atherosclerosis; autoimmune diseases including but not limited to systemic lupus erythematosus (SLE), lupus nephritis, autoimmune thyroiditis, multiple sclerosis, some forms of diabetes, and Reynaud's syndrome; tissue or organ transplant rejection disorders including but not limited to graft versus host disease (GVHD) and allograft rejection; chronic or acute glomerulonephritis; inflammatory bowel diseases including but not limited to Crohn's disease, ulcerative colitis and necrotizing enterocolitis; inflammatory dermatitis including but not limited to contact dermatitis, atopic dermatitis, psoriasis, and urticaria; fever and myalgias due to infection; central or peripheral nervous system inflammatory conditions including but not limited to meningitis (e.g., acute purulent meningitis), encephalitis, and brain or spinal cord injury due to minor trauma; Sjorgren's syndrome; diseases involving leukocyte diapedesis; alcoholic hepatitis; bacterial pneumonia; community acquired pneumonia (CAP); Pneumocystis carinii pneumonia (PCP); antigen-antibody complex mediated diseases; hypovolemic shock; Type I diabetes mellitus; acute and delayed hypersensitivity; disease states due to leukocyte dyscrasia and metastasis; thermal injury; granulocyte transfusion associated syndromes; cytokine-induced toxicity; stroke; pancreatitis; myocardial infarction, respiratory syncytial virus (RSV) infection; and spinal cord injury.

It will be appreciated that the treatment methods of the invention are useful in the fields of human medicine and veterinary medicine. Thus, the individual to be treated may be a mammal, preferably human, or other animals. For veterinary purposes, individuals include but are not limited to farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

The ability of the PI3Kδ selective inhibitors of the invention to treat arthritis can be demonstrated in a murine collagen-induced arthritis model [Kakimoto, et al., Cell. Immunol., 142:326-337 (1992)], in a rat collagen-induced arthritis model [Knoerzer, et al., Toxicol. Pathol., 25:13-19-(1997)], in a rat adjuvant arthritis model [Halloran, et al., Arthritis Rheum., 39:810-819 (1996)], in a rat streptococcal cell wall-induced arthritis model [Schimmer, et al., J. Immunol., 160:1466-1477 (1998)], or in a SCID-mouse human rheumatoid arthritis model [Oppenheimer-Marks, et al., J. Clin. Invest., 101: 1261-1272(1998)]. The ability of the PI3Kδ selective inhibitors to treat Lyme arthritis can be demonstrated according to the method of Gross, et al., Science, 218:703-706, (1998).

The ability of the PI3Kδ selective inhibitors to treat asthma can be demonstrated in a murine allergic asthma model according to the method of Wegner, et al., Science, 247:456-459 (1990), or in a murine non-allergic asthma model according to the method of Bloemen, et al, Am. J. Respir. Crit. Care Med., 153:521-529 (1996).

The ability of the PI3Kδ selective inhibitors to treat inflammatory lung injury can be demonstrated in a murine oxygen-induced lung injury model according to the method of Wegner, et al., Lung, 170:267-279 (1992), in a murine immune complex-induced lung injury model according to the method of Mulligan, et al., J. Immunol., 154:1350-1363 (1995), or in a murine acid-induced lung injury model according to the method of Nagase, et al., Am. J. Respir. Crit. Care Med., 154:504-510(1996).

The ability of the PI3Kδ selective inhibitors to treat inflammatory bowel disease can be demonstrated in a murine chemical-induced colitis model according to the method of Bennett, et al., J. Pharmacol. Exp. Ther., 280:988-1000 (1997).

The ability of the PI3Kδ selective inhibitors to treat autoimmune diabetes can be demonstrated in an NOD mouse model according to the method of Hasagawa, et al., Int. Immunol., 6:831-838 (1994), or in a murine streptozotocin-induced diabetes model according to the method of Herrold, et al., Cell Immunol., 157:489-500 (1994).

The ability of the PI3Kδ selective inhibitors to treat inflammatory liver injury can be demonstrated in a murine liver injury model according to the method of Tanaka, et al., J. Immunol., 151:5088-5095 (1993).

The ability of the PI3Kδ selective inhibitors to treat inflammatory glomerular injury can be demonstrated in a rat nephrotoxic serum nephritis model according to the method of Kawasaki, et al., J. Immunol., 150: 1074-1083 (1993).

The ability of the PI3Kδ selective inhibitors to treat radiation-induced enteritis can be demonstrated in a rat abdominal irradiation model according to the method of Panes, et al., Gastroenterology, 108:1761-1769 (1995).

The ability of the PI3Kδ selective inhibitors to treat radiation pneumonitis can be demonstrated in a murine pulmonary irradiation model according to the method of Hallahan, et al., Proc. Natl. Acad. Sci (USA), 94:6432-6437 (1997).

The ability of the PI3Kδ selective inhibitors to treat reperfusion injury can be demonstrated in the isolated heart according to the method of Tamiya, et al., Immunopharmacology, 29:53-63 (1995), or in the anesthetized dog according to the model of Hartman, et al., Cardiovasc. Res., 30:47-54 (1995).

The ability of the PI3Kδ selective inhibitors to treat pulmonary reperfusion injury can be demonstrated in a rat lung allograft reperfusion injury model according to the method of DeMeester, et al., Transplantation, 62:1477-1485 (1996), or in a rabbit pulmonary edema model according to the method of Horgan, et al., Am. J. Physiol., 261:H1578-H1584 (1991).

The ability of the PI3Kδ selective inhibitors to treat stroke can be demonstrated in a rabbit cerebral embolism stroke model according to the method of Bowes, et al., Exp. Neurol., 119:215-219 (1993), in a rat middle cerebral artery ischemia-reperfusion model according to the method of Chopp, et al., Stroke, 25:869-875 (1994), or in a rabbit reversible spinal cord ischemia model according to the method of Clark, et al., Neurosurg., 75:623-627 (1991). The ability of the PI3Kδ inhibitors to treat cerebral vasospasm can be demonstrated in a rat experimental vasospasm model according to the method of Oshiro, et al., Stroke, 28:2031-2038 (1997).

The ability of the PI3Kδ selective inhibitors to treat peripheral artery occlusion can be demonstrated in a rat skeletal muscle ischemia/reperfusion model according to the method of Gute, et al., Mol. Cell Biochem., 179:169-187 (1998).

The ability of the PI3Kδ selective inhibitors to treat graft rejection can be demonstrated in a murine cardiac allograft rejection model according to the method of Isobe, et al., Science, 255:1125-1127 (1992), in a murine thyroid gland kidney capsule model according to the method of Talento, et al., Transplantation, 55:418-422 (1993), in a cynomolgus monkey renal allograft model according to the method of Cosimi, et al., J. Immunol., 144:4604-4612 (1990), in a rat nerve allograft model according to the method of Nakao, et al., Muscle Nerve, 18:93-102 (1995), in a murine skin allograft model according to the method of Gorczynski and Wojcik, J. Immunol., 152:2011-2019 (1994), in a murine corneal allograft model according to the method of He, et al., Opthalmol. Vis. Sci., 35:3218-3225 (1994), or in a xenogeneic pancreatic islet cell transplantation model according to the method of Zeng, et al., Transplantation, 58:681-689 (1994).

The ability of the PI3Kδ selective inhibitors to treat graft-versus-host disease (GVHD) can be demonstrated in a murine lethal GVHD model according to the method of Harning, et al., Transplantation, 52:842-845 (1991).

The ability of the PI3Kδ selective inhibitors to treat cancers can be demonstrated in a human lymphoma metastasis model (in mice) according to the method of Aoudjit, et al., J. Immunol., 161:2333-2338 (1998).

As previously described, the term “PI3Kδ selective inhibitor” generally refers to a compound that inhibits the activity of the PI3Kδ isozyme more effectively than other isozymes of the PI3K family. The relative efficacies of compounds as inhibitors of an enzyme activity (or other biological activity) can be established by determining the concentrations at which each compound inhibits the activity to a predefined extent and then comparing the results. Typically, the preferred determination is the concentration that inhibits 50% of the activity in a biochemical assay, i.e., the 50% inhibitory concentration or “IC₅₀.” IC₅₀ determinations can be accomplished using conventional techniques known in the art. In general, an IC₅₀ can be determined by measuring the activity of a given enzyme in the presence of a range of concentrations of the inhibitor under study. The experimentally obtained values of enzyme activity then are plotted against the inhibitor concentrations used. The concentration of the inhibitor that shows 50% enzyme activity (as compared to the activity in the absence of any inhibitor) is taken as the IC₅₀ value. Analogously, other inhibitory concentrations can be defined through appropriate determinations of activity. For example, in some settings it can be desirable to establish a 90% inhibitory concentration, i.e., IC₉₀, etc.

Accordingly, a PI3Kδ selective inhibitor alternatively can be understood to refer to a compound that exhibits a 50% inhibitory concentration (IC₅₀) with respect to PI3Kδ that is at least 10-fold, in another aspect at least 20-fold, and in another aspect at least 30-fold, lower than the IC₅₀ value with respect to any or all of the other class I PI3K family members. In an alternative embodiment of the invention, the term PI3Kδ selective inhibitor can be understood to refer to a compound that exhibits an IC₅₀ with respect to PI3Kδ that is at least 50-fold, in another aspect at least 100-fold, in an additional aspect at least 200-fold, and in yet another aspect at least 500-fold, lower than the IC₅₀ with respect to any or all of the other PI3K class I family members. A PI3Kδ selective inhibitor is typically administered in an amount such that it selectively inhibits PI3Kδ activity, as described above.

Any selective inhibitor of PI3Kδ activity, including but not limited to small molecule inhibitors, peptide inhibitors, non-peptide inhibitors, naturally occurring inhibitors, and synthetic inhibitors, may be used in the methods. Suitable PI3Kδ selective inhibitors have been described in U.S. patent Publication 2002/161014 to Sadhu, et al. and Knight, et al., Bioorganic & Medicinal Chemistry, 12:4749-4759 (2004), the entire disclosures of which are hereby incorporated herein by reference. Compounds that compete with a PI3Kδ selective inhibitor compound described herein for binding to PI3Kδ and selectively inhibit PI3Kδ are also contemplated for use in the methods of the invention. Methods of identifying compounds which competitively bind with PI3Kδ, with respect to the PI3Kδ selective inhibitor compounds specifically provided herein, are well known in the art [see, e.g., Coligan, et al., Current Protocols in Protein Science, A.5A. 15-20, vol.3 (2002)]. In view of the above disclosures, therefore, PI3Kδ selective inhibitor embraces the specific PI3Kδ selective inhibitor compounds disclosed herein, compounds having similar inhibitory profiles, and compounds that compete with the such PI3Kδ selective inhibitor compounds for binding to PI3Kδ, and in each case, conjugates and derivatives thereof.

The methods of the invention may be applied to cell populations in vivo or ex vivo. “In vivo” means within a living individual, as within an animal or human. In this context, the methods of the invention may be used therapeutically or prophylactically in an individual, as described infra.

“Ex vivo” means outside of a living individual. Examples of ex vivo cell populations include in vitro cell cultures and biological samples including but not limited to fluid or tissue samples obtained from individuals. Such samples may be obtained by methods well known in the art. Exemplary biological fluid samples include blood, cerebrospinal fluid, urine, saliva. Exemplary tissue samples include tumors and biopsies thereof. In this context, the invention may be used for a variety of purposes, including therapeutic and experimental purposes. For example, the invention may be used ex vivo to determine the optimal schedule and/or dosing of administration of a PI3Kδ selective inhibitor for a given indication, cell type, individual, and other parameters. Information gleaned from such use may be used for experimental or diagnostic purposes or in the clinic to set protocols for in vivo treatment. Other ex vivo uses for which the invention may be suited are described below or will become apparent to those skilled in the art.

The methods in accordance with the invention may include administering a PI3Kδ selective inhibitor with one or more other agents that either enhance the activity of the inhibitor or compliment its activity or use in treatment. Such additional factors and/or agents may produce an augmented or even synergistic effect when administered with a PI3Kδ selective inhibitor, or minimize side effects.

In one embodiment, the methods of the invention may include administering formulations comprising a PI3Kδ selective inhibitor of the invention with a particular cytokine, lymphokine, other hematopoietic factor, thrombolytic or anti-thrombotic factor, or anti-inflammatory agent before, during, or after administration of the PI3Kδ selective inhibitor. One of ordinary skill can easily determine if a particular cytokine, lymphokine, hematopoietic factor, thrombolytic of anti-thrombotic factor, and/or anti-inflammatory agent enhances or compliments the activity or use of the PI3Kδ selective inhibitors in treatment.

More specifically, and without limitation, the methods of the invention may comprise administering a PI3Kδ selective inhibitor with one or more of TNF, IL-1, IL-2, IL-3, IL4, IL-5, IL-6, IL-7, IL-8, IL-9, IL-10, IL-11, IL-12, IL-13, IL-14, IL-15, IL-16, IL-17, IL-18, IFN, G-CSF, Meg-CSF, GM-CSF, thrombopoietin, stem cell factor, and erythropoietin. Compositions in accordance with the invention may also include other known angiopoietins such as Ang-2, Ang4, and Ang-Y, growth factors such as bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, brain derived neurotrophic factor, ciliary neutrophic factor, ciliary neutrophic factor receptor a, cytokine-induced neutrophil chemotactic factor 1, cytokine-induced neutrophil chemotactic factor 2α, cytokine-induced neutrophil chemotactic factor 2β, β endothelial cell growth factor, endothelin 1, epidermal growth factor, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor acidic, fibroblast growth factor basic, glial cell line-derived neutrophic factor receptor al, glial cell line-derived neutrophic factor receptor a2, growth related protein, growth related protein a, growth related protein β, growth related protein γ, heparin binding epidermal growth factor, hepatocyte growth factor, hepatocyte growth factor receptor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor, nerve growth factor receptor, neurotrophin-3, neurptrophin-4, placenta growth factor, placenta growth factor 2, platelet derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor a, platelet derived growth factor receptor β, pre-B cell growth stimulating factor, stem cell factor, stem cell factor receptor, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor β binding protein III, tumor necrosis factor receptor type I, tumor necrosis factor receptor type II, urokinase-type plasminogen activator receptor, and chimeric proteins and biologically or immunologically active fragments thereof.

Methods of the invention contemplate use of PI3Kδ selective inhibitor compound having formula (I) or pharmaceutically acceptable salts and solvates thereof:

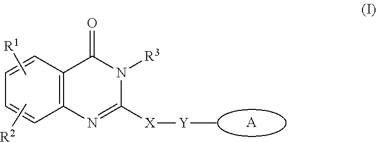

wherein A is an optionally substituted monocyclic or bicyclic ring system containing at least two nitrogen atoms, and at least one ring of the system is aromatic;

X is selected from the group consisting of C(R^{b})₂, CH₂CHR^{b}, and CH═C(R^{b});

Y is selected from the group consisting of null, S, SO, SO₂, NH, 0, C(═O), OC(═O), C(═O)O, and NHC(═O)CH₂S;

R¹ and R², independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═(O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

or R¹ and R² are taken together to form a 3- or 4-membered alkylene or alkenylene chain component of a 5- or 6-membered ring, optionally containing at least one heteroatom;

R³ is selected from the group consisting of optionally substituted hydrogen, C₁₋₆alkyl, C₃₋₈cycloalkyl, C₃₋₈heterocycloalkyl, C₁₋₄alkylenecycloalkyl, C₂₋₆alkenyl, C₁₋₃alkylenearyl, arylC₁₋₃alkyl, C(═O)R^{a}, aryl, heteroaryl, C(═O)OR^{a}, C(═O)N(R^{a})₂, C(═S)N(R^{a})₂, SO₂R^{a}, SO₂N(R^{a})₂, S(═O)R^{a}, S(═O)N(R^{a})₂, C(═O)NR^{a}C₁₋₄alkyleneHet, C(═O)C₁₋₄alkylenearyl, C(═O)C₁₋₄alkyleneheteroaryl, C₁₋₄alkylenearyl optionally substituted with one or more of halo, SO₂N(R^{a})₂, N(R^{a})₂, C(═O)OR^{a}, NR^{a}SO₂CF₃, CN, NO₂, C(═O)R^{a}, OR^{a}, C₁₋₄alkyleneN(R^{a})₂, and OC₁₋₄alkyleneN(R^{a})₂, C₁₋₄alkyleneheteroaryl, C₁₋₄alkyleneHet, C₁₋₄alkyleneC(═O)C₁₋₄alkylenearyl, C₁₋₄alkyleneC(═O)C₁₋₄alkyleneheteroaryl, C₁₋₄alkyleneC(═O)Het, C₁₋₄alkyleneC(═O)N(R^{a})₂, C₁₋₄alkyleneOR^{a}, C₁₋₄alkyleneNR^{a}C(═O)R^{a}, C₁₋₄alkyleneOC₁₋₄alkyleneOR^{a}, C₁₋₄alkyleneN(R^{a})₂, C₁₋₄alkyleneC(═O)OR^{a}, and C₁₋₄alkyleneOC₁₋₄alkyleneC(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{b} is selected from the group consisting of hydrogen, C₁₋₆alkyl, heteroC₁₋₃alkyl, C₁₋₃alkyleneheteroC₁₋₃alkyl, arylheteroC₁₋₃alkyl, aryl, heteroaryl, arylC₁₋₃alkyl, heteroarylC₁₋₃alkyl, C₁₋₃alkylenearyl, and C₁₋₃alkyleneheteroaryl;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

As used herein, the term “alkyl” is defined as straight chained and branched hydrocarbon groups containing the indicated number of carbon atoms, typically methyl, ethyl, and straight chain and branched propyl and butyl groups. The hydrocarbon group can contain up to 16 carbon atoms, for example, one to eight carbon atoms. The term “alkyl” includes “bridged alkyl,” i.e., a C₆-C₁₆ bicyclic or polycyclic hydrocarbon group, for example, norbornyl, adamantyl, bicyclo[2.2.2]octyl, bicyclo[2.2.1]heptyl, bicyclo[3.2.1]octyl, or decahydronaphthyl. The term “cycloalkyl” is defined as a cyclic C₃-C₈ hydrocarbon group, e.g., cyclopropyl, cyclobutyl, cyclohexyl, and cyclopentyl.

The term “alkenyl” is defined identically as “alkyl,” except for containing a carbon-carbon double bond. “Cycloalkenyl” is defined similarly to; cycloalkyl, except a carbon-carbon double bond is present in the ring.

The term “alkylene” is defined as an alkyl group having a substituent. For example, the term “C₁₋₃alkylenearyl” refers to an alkyl group containing one to three carbon atoms, and substituted with an aryl group.

The term “heteroC₁₋₃alkyl” is defined as a C₁₋₃alkyl group further containing a heteroatom selected from O, S, and NR^{a}. For example, —CH₂OCH₃ or —H₂CH₂SCH₃. The term “arylheteroC₁₋₃alkyl” refers to an aryl group having a heteroC₁₋₃alkyl substituent.

The term “halo” or “halogen” is defined herein to include fluorine, bromine, chlorine, and iodine.

The term “aryl,” alone or in combination, is defined herein as a monocyclic or polycyclic aromatic group, e.g., phenyl or naphthyl. Unless otherwise indicated, an “aryl” group can be unsubstituted or substituted, for example, with one or more, and in particular one to three, halo, alkyl, phenyl, hydroxyalkyl, alkoxy, alkoxyalkyl, haloalkyl, nitro, and amino. Exemplary aryl groups include phenyl, naphthyl, biphenyl, tetrahydronaphthyl, chlorophenyl, fluorophenyl, aminophenyl, methylphenyl, methoxyphenyl, trifluoromethylphenyl, nitrophenyl, carboxyphenyl, and the like. The terms “arylC₁₋₃ alkyl” and “heteroarylC₁₋₃alkyl” are defined as an aryl or heteroaryl group having a C₁₋₃alkyl substituent.

The term “heteroaryl” is defined herein as a monocyclic or bicyclic ring system containing one or two aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, and amino. Examples of heteroaryl groups include thienyl, furyl, pyridyl, oxazolyl, quinolyl, isoquinolyl, indolyl, triazolyl, isothiazolyl, isoxazolyl, imidizolyl, benzothiazolyl, pyrazinyl, pyrimidinyl, thiazolyl, and thiadiazolyl.

The term “Het” is defined as monocyclic, bicyclic, and tricyclic groups containing one or more heteroatoms selected from the group consisting of oxygen, nitrogen, and sulfur. A “Het” group also can contain an oxo group (═O) attached to the ring. Nonlimiting examples of Het groups include 1,3-dioxolane, 2-pyrazoline, pyrazolidine, pyrrolidine, piperazine, a pyrroline, 2H-pyran, 4H-pyran, morpholine, thiopholine, piperidine, 1,4-dithiane, and 1,4-dioxane.

Alternatively, the PI3Kδ selective inhibitor may be a compound having formula (II) or pharmaceutically acceptable salts and solvates thereof:

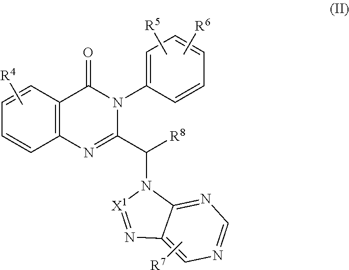

wherein R⁴, R⁵, R⁶, and R⁷, independently, are selected from the group consisting of hydrogen, C₁₋₆alkyl, aryl, heteroaryl, halo, NHC(═O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂Calkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃ alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R⁸ is selected from the group consisting of hydrogen, C₁₋₆alkyl, halo, CN, C(═O)R^{a}, and C(═O)OR^{a};

X¹ is selected from the group consisting of CH (i.e., a carbon atom having a hydrogen atom attached thereto) and nitrogen;

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

The PI3Kδ selective inhibitor may also be a compound having formula (III) or pharmaceutically acceptable salts and solvates thereof:

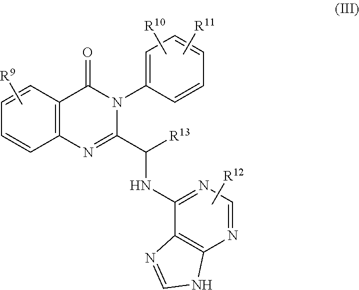

wherein R⁹, R¹⁰, R¹¹, and R¹², independently, are selected from the group consisting of hydrogen, amino, C₁₋₆alkyl, aryl, heteroaryl, halo, NHC(═(O)C₁₋₃alkyleneN(R^{a})₂, NO₂, OR^{a}, CF₃, OCF₃, N(R^{a})₂, CN, OC(═O)R^{a}, C(═O)R^{a}, C(═O)OR^{a}, arylOR^{b}, Het, NR^{a}C(═O)C₁₋₃alkyleneC(═O)OR^{a}, arylOC₁₋₃alkyleneN(R^{a})₂, arylOC(═O)R^{a}, C₁₋₄alkyleneC(═O)OR^{a}, OC₁₋₄alkyleneC(═O)OR^{a}, C₁₋₄alkyleneOC₁₋₄alkyleneC(═O)OR^{a}, C(═O)NR^{a}SO₂R^{a}, C₁₋₄alkyleneN(R^{a})₂, C₂₋₆alkenyleneN(R^{a})₂, C(═O)NR^{a}C₁₋₄alkyleneOR^{a}, C(═O)NR^{a}C₁₋₄alkyleneHet, OC₂₋₄alkyleneN(R^{a})₂, OC₁₋₄alkyleneCH(OR^{b})CH₂N(R^{a})₂, OC₁₋₄alkyleneHet, OC₂₋₄alkyleneOR^{a}, OC₂₋₄alkyleneNR^{a}C(═O)OR^{a}, NR^{a}C₁₋₄alkyleneN(R^{a})₂, NR^{a}C(═O)R^{a}, NR^{a}C(═O)N(R^{a})₂, N(SO₂C₁₋₄alkyl)₂, NR^{a}(SO₂C₁₋₄alkyl), SO₂N(R^{a})₂, OSO₂CF₃, C₁₋₃alkylenearyl, C₁₋₄alkyleneHet, C₁₋₆alkyleneOR^{b}, C₁₋₃alkyleneN(R^{a})₂, C(═O)N(R^{a})₂, NHC(═O)C₁₋₃alkylenearyl, C₃₋₈cycloalkyl, C₃₋₈heterocycloalkyl, arylOC₁₋₃alkyleneN(R^{a})₂, arylOC(═O)R^{b}, NHC(═O)C₁₋₃alkyleneC₃₋₈heterocycloalkyl, NHC(═O)C₁₋₃alkyleneHet, OC₁₋₄alkyleneOC₁₋₄alkyleneC(═O)OR^{b}, C(═O)C₁₋₄alkyleneHet, and NHC(═O)haloC₁₋₆alkyl;

R¹³ is selected from the group consisting of hydrogen, C₁₋₆alkyl, halo, CN, C(═O)R^{a}, and C(═O)OR^{a};

R^{a} is selected from the group consisting of hydrogen, C₁₋₆alkyl, C₃₋₈cycloalkyl, C₃₋₈heterocycloalkyl, C₁₋₃alkyleneN(R^{c})₂, aryl, arylC₁₋₃alkyl, C₁₋₃alkylenearyl, heteroaryl, heteroarylC₁₋₃alkyl, and C₁₋₃alkyleneheteroaryl;

or two R^{a} groups are taken together to form a 5- or 6-membered ring, optionally containing at least one heteroatom;

R^{c} is selected from the group consisting of hydrogen, C₁₋₆alkyl, C₃₋₈cycloalkyl, aryl, and heteroaryl; and,

Het is a 5- or 6-membered heterocyclic ring, saturated or partially or fully unsaturated, containing at least one heteroatom selected from the group consisting of oxygen, nitrogen, and sulfur, and optionally substituted with C₁₋₄alkyl or C(═O)OR^{a}.

More specifically, representative PI3Kδ selective inhibitors in accordance with the foregoing chemical formulae include but are not limited to
2-(6-aminopurin-9-ylmethyl)-3-(2-chlorophenyl)-6,7-dimethoxy-3-H-quinazolin-4-one;
2-(6-aminopurin-o-ylmethyl)-6-bromo-3-(2-chlorophenyl)-3H-quinazolin-4-one;
2-(6-aminopurin-o-ylmethyl)-3-(2-chlorophenyl)-7-fluoro-3H-quinazolin-4-one;
2-(6-aminopurin-9-ylmethyl)-6-chloro-3-(2-chlorophenyl)-3H-quinazolin-4-one;
2-(6-aminopurin-9-ylmethyl)-3-(2-chlorophenyl)-5-fluoro-3H-quinazolin-4-one;
2-(6-aminopurin-o-ylmethyl)-5-chloro-3-(2-chloro-phenyl)-3H-quinazolin-4-one;
2-(6-aminopurin-9-ylmethyl)-3-(2-chlorophenyl)-5-methyl-3H-quinazolin-4-one;
2-(6-aminopurin-9-ylmethyl)-8-chloro-3-(2-chlorophenyl)-3H-quinazolin-4-one;
2-(6-aminopurin-9-ylmethyl)-3-biphenyl-2-yl-5-chloro-3H-quinazolin-4-one;
5-chloro-2-(9H-purin-6-ylsulfanylmethyl)-3-o-tolyl-3H-quinazolin-4-one;
5-chloro-3-(2-fluorophenyl)-2-(9H-purin-6-yl-sulfanylmethyl)-3H-quinazolin-4-one;
2-(6-aminopurin-9-ylmethyl)-5-chloro-3-(2-fluorophenyl)-3 H-quinazolin-4-one ;
3-biphenyl-2-yl-5-chloro-2-(9H-purin-6-ylsulfanylmethyl)-3H-quinazolin-4-one;
5-chloro-3-(2-methoxyphenyl)-2-(9H-purin-6-yl-sulfanylmethyl)-3H-quinazolin-4-one;
3-(2-chlorophehyl)-5-fluoro-2-(9H-purin-6-yl-sulfanylmethyl)-3H-quinazolin-4-one;
3-(2-chlorophenyl)-6,7-dimethoxy-2-(9H-purin-6-yl-sulfanylmethyl)-3H-quinazolin-4-one;
6-bromo-3-(2-chlorophenyl )-2-(9H-purin-6-yl-sulfanylmethyl)-3H-quinazolin-4-one ;
3-(2-chlorophenyl)-8-trifluoromethyl-2-(9H-purin-6-ylsulfanylmethyl)-3H-quinazolin-4-one;
3-(2-chlorophenyl)-2-(9H-purin-6-ylsulfanylmethyl)-3H-benzo[g]quinazolin-4-one;
6-chloro-3-(2-chlorophenyl)-2-(9H-purin-6-yl-sulfanylmethyl)-3H-quinazolin-4-one;
8-chloro-3-(2-chlorophenyl)-2-(9H-purin-6-yl-sulfanylmethyl)-3H-quinazolin-4-one;
3-(2-chlorophenyl)-7-fluoro-2-(9H-purin-6-yl-sulfanylmethyl)-3H-quinazolin-4-one;
3-(2-chlorophenyl)-7-nitro-2-(9H-purin-6-yl-sulfanylmethyl)-3H-quinazolin-4-one;
3-(2-chlorophenyl)-6-hydroxy-2-(9H-purin-6-yl-sulfanylmethyl)-3H-quinazolin-4-one;
5-chloro-3-(2-chlorophenyl)-2-(9H-purin-6-yl-sulfanylmethyl)-3H-quinazolin-4-one;
3-(2-chlorophenyl)-5-methyl-2-(9H-purin-6-yl-sulfanylmethyl)-3H-quinazolin-4-one;
3-(2-chlorophenyl)-6,7-difluoro-2-(9H-purin-6-yl-sulfanylmethyl)-3H-quinazolin-4-one;
3-(2-chlorophenyl)-6-fluoro-2-(9H-purin-6-yl-sulfanylmethyl)-3H-quinazolin-4-one;
2-(6-aminopurin-9-ylmethyl)-3-(2-isopropylphenyl)-5-methyl-3H-quinazolin-4-one;
2-(6-aminopurin-9-ylmethyl)-5-methyl-3-o-tolyl-3H-quinazolin-4-one;
3-(2-fluorophenyl)-5-methyl-2-(9H-purin-6-yl-sulfanylmethyl)-3H-quinazolin-4-one ;
2-(6-aminopurin-9-ylmethyl)-5-chloro-3-o-tolyl-3H-quinazolin-4-one;
2-(6-aminopurin-9-ylmethyl)-5-chloro-3-(2-methoxy-phenyl)-3H-quinazolin-4-one;
2-(2-amino-9H-purin-6-ylsulfanylmethyl)-3-cyclopropyl-5-methyl-3H-quinazolin-4-one;
3-cyclopropylmethyl-5-methyl-2-(9-H-purin-6-ylsulfanylmethyl)-3H-quinazolin-4-one;
2-(6-aminopurin-9-ylmethyl)-3-cyclopropylmethyl-5-methyl-3H-quinazolin-4-one;
2-(2-amino-9H-purin-6-ylsulfanylmethyl)-3-cyclopropylmethyl-5-methyl-3H-quinazolin-4-one;
5-methyl-3-phenethyl-2-(9H-purin-6-ylsulfanylmethyl)-3H-quinazolin-4-one;
2-(2-amino-9H-purin-6-ylsulfanylmethyl)-5-methyl-3-phenethyl-3H-quinazolin-4-one;
3-cyclopentyl-5-methyl-2-(9H-purin-6-ylsulfanylmethyl)-3H-quinazolin-4-one;
2-(6-aminopurin-9-ylmethyl)-3-cyclopentyl-5-methyl-3H-quinazolin-4-one;
3-(2-chloropyridin-3-yl)-5-methyl-2-(9H-purin-6-ylsulfanylmethyl)-3H-quinazolin-4-one;
2-(6-aminopurin-9-ylmethyl)-3-(2-chloropyridin-3-yl)-5-methyl-3H-quinazolin-4-one;
3-methyl-4-[5-methyl-4-oxo-2-(9H-purin-6-ylsulfanylmethyl)-4H-quinazolin-3-yl]-benzoic acid;
3-cyclopropyl-5-methyl-2-(9H-purin-6-ylsulfanylmethyl)-3H-quinazolin-4-one;
2-(6-aminopurin-9-ylmethyl)-3-cyclopropyl-5-methyl-3H-quinazolin-4-one;
5-methyl-3-(4-nitrobenzyl)-2-(9H-purin-6-ylsulfanylmethyl)-3H-quinazolin-4-one;
3-cyclohexyl-5-methyl-2-(9H-purin-6-ylsulfanylmethyl)-3H-quinazolin-4-one;
2-(6-aminopurin-9-ylmethyl-3-cyclohexyl-5-methyl-3H-quinazolin-4-one;
2-(2-amino-9H-purin-6-ylsulfanylmethyl)-3-cyclohexyl-5-methyl-3H-quinazolin-4-one;
5-methyl-3-(E-2-phenylcyclopropyl)-2-(9H-purin-6-ylsulfanylmethyl)-3H-quinazolin-4-one;
3-(2-chlorophenyl)-5-fluoro-2-[(9H-purin-6-ylamino)methyl]-3H-quinazolin-4-one;
2-[(2-amino-9H-purin-6-ylamino)methyl]-3-(2-chlorophenyl)-5-fluoro-3H-quinazolin-4-one;
5-methyl-2-[(9H-purin-6-ylamino)methyl]-3-o-tolyl-3H-quinazolin-4-one;
2-[(2-amino-9H-purin-6-ylamino)methyl]-5-methyl-3-o-tolyl-3H-quinazolin-4-one;
2-[(2-fluoro-9H-purin-6-ylamino)methyl]-5-methyl-3-o-tolyl-3H-quinazolin-4-one;
(2-chlorophenyl)-dimethylamino-(9H-purin-6-ylsulfanylmethyl)-3H-quinazolin-4-4-one;
5-(2-benzyloxyethoxy)-3-(2-chlorophenyl)-2-(9H-purin-6-ylsulfanylmethyl)-3H-quinazolin-4-one;
6-aminopurine-9-carboxylic acid 3-(2-chlorophenyl)-5-fluoro-4-oxo-3,4-dihydro-quinazolin-2-ylmethyl ester;
N-[3-(2-chlorophenyl)-5-fluoro-4-oxo-3,4-dihydro-quinazolin-2-ylmethyl]-2-(9H-purin-6-ylsulfanyl)-acetamide;
2-[1-(2-fluoro-9H-purin-6-ylamino)ethyl]-5-methyl-3-o-tolyl-3H-quinazolin-4-one;
5-methyl-2-[1-(9H-purin-6-ylamino)ethyl]-3-o-tolyl-3H-quinazolin-4-one;
2-(6-dimethylaminopurin-9-ylmethyl)-5-methyl-3-o-tolyl-3H-quinazolin-4-one;
5-methyl-2-(2-methyl-6-oxo-1,6-dihydro-purin-7-ylmethyl)-3-o-tolyl-3H-quinazolin-4-one;
5-methyl-2-(2-methyl-6-oxo-1,6-dihydro-purin-9-ylmethyl)-3-o-tolyl-3H-quinazolin-4-one;
2-(amino-dimethylaminopurin-9-ylmethyl)-5-methyl-3-o-tolyl-3H-quinazolin-4-one;
2-(2-amino-9H-purin-6-ylsulfanylmethyl)-5-methyl-3-o-tolyl-3H-quinazolin-4-one;
2-(4-amino-1,3,5-triazin-2-ylsulfanylmethyl)-5-methyl-3-o-tolyl-3H-quinazolin-4-one;
5-methyl-2-(7-methyl-7H-purin-6-ylsulfanylmethyl)-3-o-tolyl-3H-quinazolin-4-one;
5-methyl-2-(2-oxo-1,2-dihydro-pyrimidin-4-ylsulfanylmethyl)-3-o-tolyl-3H-quinazolin-4-one;
5-methyl-2-purin-7-ylmethyl-3-o-tolyl-3H-quinazolin-4-one;
5-methyl-2-purin-9-ylmethyl-3-o-tolyl-3H-quinazolin-4-one;
5-methyl-2-(9-methyl-9H-purin-6-ylsulfanylmethyl)-3-o-tolyl-3H-quinazolin-4-one;
2-(2,6-diamino-pyrimidin-4-ylsulfanylmethyl)-5-methyl-3-o-tolyl-3H-quinazolin-4-one;
5-methyl-2-(5-methyl-[1,2,4]triazolo[1,5-a]pyrimidin-7-ylsulfanylmethyl)-3-o-tolyl-3H-quinazolin-4-one;
5-methyl-2-(2-methylsulfanyl-9H-purin-6-ylsulfanylmethyl)-3-o-tolyl-3H-quinazolin-4-one;
2-(2-hydroxy-9H-purin-6-ylsulfanylmethyl)-5-methyl-3-o-tolyl-3H-quinazolin-4-one;
5-methyl-2-(1-methyl-1H-imidazol-2-ylsulfanylmethyl)-3-o-tolyl-3H-quinazolin-4-one;
5-methyl-3-o-tolyl-2-(1H-[1,2,4]triazol-3-ylsulfanylmethyl)-3H-quinazolin-4-one;
2-(2-amino-6-chloro-purin-9-ylmethyl)-5-methyl-3-o-tolyl-3H-quinazolin-4-one;
2-(6-aminopurin-7-ylmethyl)-5-methyl-3-o-tolyl-3H-quinazolin-4-one;
2-(7-amino-1,2,3-triazolo[4,5-d]pyrimidin-3-yl-methyl)-5-methyl-3-o-tolyl-3H-quinazolin-4-one; 2-(7-amino-1,2,3-triazolo[4,5-d]pyrimidin-1-yl-methyl)-5-methyl-3-o-tolyl-3H-quinazolin-4-one;
2-(6-amino-9H-purin-2-ylsulfanylmethyl)-5-methyl-3-o-tolyl-3H-quinazolin-4-one;
2-(2-amino-6-ethylamino-pyrimidin-4-ylsulfanylmethyl)-5-methyl-3-o-tolyl-3H-quinazolin-4-one; 2-(3-amino-5-methylsulfanyl-1,2,4-triazol-1-yl-methyl)-5-methyl-3-o-tolyl-3H-quinazolin-4-one;
2-(5-amino-3-methylsulfanyl-1,2,4-triazol-1-ylmethyl)-5-methyl-3-o-tolyl-3H-quinazolin-4-one;
5-methyl-2-(6-methylaminopurin-9-ylmethyl)-3-o-tolyl-3H-quinazolin-4-one;
2-(6-benzylaminopurin-9-ylmethyl)-5-methyl-3-o-tolyl-3H-quinazolin-4-one;
2-(2,6-diaminopurin-9-ylmethyl)-5-methyl-3-o-tolyl-3H-quinazolin-4-one;
5-methyl-2-(9H-purin-6-ylsulfanylmethyl)-3-o-tolyl-3H-quinazolin-4-one;
3-isobutyl-5-methyl-2-(9H-purin-6-ylsulfanylmethyl)-3H-quinazolin-4-one; N-{2-[5-Methyl-4-oxo-2-(9H-purin-6-ylsulfanylmethyl)-4H-quinazolin-3-yl]-phenyl}-acetamide;
5-methyl-3-(E-2-methyl-cyclohexyl)-2-(9H-purin-6-ylsulfanylmethyl)-3H-quinazolin-4-one;
2-[5-methyl-4-oxo-2-(9H-purin-6-ylsulfanylmethyl)-4H-quinazolin-3-yl]-benzoic acid-3-{2-[(2-dimethylaminoethyl)methylaminolphenyl}-5-methyl-2-(9H-purin-6-ylsulfanylmethyl)-3H-quinazolin-4-one;
3-(2-chlorophenyl)-5-methoxy-2-(9H-purin-6-ylsulfanylmethyl)-3H-quinazolin-4-one;
3-(2-chlorophenyl)-5-(2-morpholin-4-yl-ethylamino)-2-(9H-purin-6-ylsulfanylmethyl )-3H-quinazolin-4-one; 3-benzyl-5-methoxy-2-(9H-purin-6-ylsulfanylmethyl)-3H-quinazolin-4-one;
2-(6-aminopurin-9-ylmethyl)-3-(2-benzyloxyphenyl)-5-methyl-3H-quinazolin-4-one;
2-(6-aminopurin-9-ylmethyl)-3-(2-hydroxyphenyl)-5-methyl-3H-quinazolin-4-one;
2-(1-(2-amino-9H-purin-6-ylamino)ethyl)-5-methyl-3-o-tolyl-3H-quinazolin-4-one;
5-methyl-2-[1-(9H-purin-6-ylamino)propyl]-3-o-tolyl-3H-quinazolin-4-one;
2-(1-(2-fluoro-9H-purin-6-ylamino)propyl)-5-methyl-3-o-tolyl-3H-quinazolin-4-one;
2-(1-(2-amino-9H-purin-6-ylamino)propyl)-5-methyl-3-o-tolyl-3H-quinazolin-4-one;
2-(2-benzyloxy-1-(9H-purin-6-ylamino)ethyl)-5-methyl-3-o-tolyl-3H-quinazolin-4-one;
2-(6-aminopurin-9-ylmethyl)-5-methyl-3-{2(2-(1-methylpyrrolidin-2-yl)-ethoxy)-phenyl}-3H-quinazolin-4-one; 2-(6-aminopurin-9-ylmethyl)-3-(2-(3-dimethylamino-propoxy)-phenyl)-5-methyl-3H-quinazolin-4-one;
2-(6-aminopurin-9-ylmethyl)-5-methyl-3-(2-prop-2-ynyloxyphenyl)-3H-quinazolin-4-one;
2-{2-(1-(6-aminopurin-9-ylmethyl)-5-methyl-4-oxo-4H-quinazolin-3-yl]-phenoxy)}-acetamide;
2-[(6-aminopurin-9-yl)methyl]-5-methyl-3-o-tolyl-3-hydroquinazolin-4-one;
3-(3,5-difluorophenyl)-5-methyl-2-[(purin-6-ylamino)methyl]-3-hydroquinazolin-4-one;
3-(2,6-dichlorophenyl)-5-methyl-2-[(purin-6-ylamino)methyl]-3-hydroquinazolin-4-one;
3-(2-fluoro-phenyl)-2-[1-(2-fluoro-9H-purin-6-ylamino)-ethyl]-5-methyl-3-hydroquinazolin-4-one;
2-[1-(6-aminopurin-9-yl)ethyl-3-(3,5-difluorophenyl)-5-methyl-3-hydroquinazolin-4-one;
2-[1-(7-amino-[1,2,3]triazolo[4,5-d]pyrimidin-3-yl)-ethyl]-3-(3,5-difluorophenyl)-5-methyl-3H-quinazolin-4-one;
5-chloro-3-(3,5-difluoro-phenyl)-2-[1-(9H-purin-6-ylamino)-propyl]-3H-quinazolin-4-one;
3-phenyl-2-[1-(9H-purin-6-ylamino)-propyl]-3H-quinazolin-4-one;
5-fluoro-3-phenyl-2-[1-(9H-purin-6-ylamino)-propyl]-3H-quinazolin-4-one;
3-(2,6-difluoro-phenyl)-5-methyl-2-[1-(9H-purin-6-ylamino)-propyl]-3H-quinazolin-4-one;
6-fluoro-3-phenyl-2-[1-(9H-purin-6-ylamino)-ethyl]-3H-quinazolin-4-one;
3-(3,5-difluoro-phenyl)-5-methyl-2-[1-(9H-purin-6-ylamino)-ethyl]-3H-quinazolin-4-one;
5-fluoro-3-phenyl-2-[1-(9H-purin-6-ylamino)-ethyl]-3H-quinazolin-4-one;
3-(2,3-difluoro-phenyl)-5-methyl-2-[1-(9H-purin-6-ylamino)-ethyl]-3H-quinazolin-4-one;
5-methyl-3-phenyl-2-[1-(9H-purin-6-ylamino)-ethyl]-3H-quinazolin-4-one;
3-(3-chloro-phenyl)-5-methyl-2-[1-(9H-purin-6-ylamino)-ethyl]-3H-quinazolin-4-one;
5-methyl-3-phenyl-2-[(9H-purin-6-ylamino)-methyl]-3H-quinazolin-4-one;
2-[(2-amino-9H-purin-6-ylamino)-methyl]-3-(3,5-difluoro-phenyl)-5-methyl-3H-quinazolin-4-one;
3-{2-[(2-diethylamino-ethyl)-methyl-amino]-phenyl}-5-methyl-2-[(9H-purin-6-ylamino)-methyl]-3H-quinazolin-4-one;
5-chloro-3-(2-fluoro-phenyl)-2-[(9H-purin-6-ylamino)-methyl]-3H-quinazolin-4-one;
5-chloro-2-[(9H-purin-6-ylamino)-methyl]-3-o-tolyl-3H-quinazolin-4-one;
5-chloro-3-(2-chloro-phenyl)-2-[(9H-purin-6-ylamino)-methyl]-3H-quinazolin-4-one;
6-fluoro-3-(3-fluoro-phenyl)-2-[1-(9H-purin-6-ylamino)-ethyl]-3H-quinazolin-4-one; and
2-[1-(2-amino-9H-purin-6-ylamino)-ethyl]-5-chloro-3-(3-fluoro-phenyl)-3H-quinazolin-4-one.
   Where a stereocenter is present, the methods can be practiced using a racemic mixture of the compounds or a specific enantiomer. In preferred embodiments where a stereocenter is present, the S-enantiomer of the above compounds is utilized. However, the methods of the invention include administration of all-possible stereoisomers and geometric isomers of the aforementioned-compounds.

Additionally, the methods include administration of PI3Kδ selective inhibitors comprising an arylmorpholine moiety [Knight, et al., Bioorganic & Medicinal Chemistry, 12:4749-4759 (2004)]. Representative PI3Kδ selective inhibitors include but are not limited to
2-morpholin-4-yl-8-o-tolyloxy-1H-quinolin-4-one;
9-bromo-7-methyl-2-morpholin-4-yl-pyrido(1,2-a)-pyrimidin-4-one;
9-benzylamino-7-methyl-2-morpholin-4-yl-pyrido-(1,2-a)-pyrimidin-4-one;
9-(3-amino-phenyl)-7-methyl-2-morpholin-4-yl-pyrido(1,2-a)pyrimidin-4-one;
9-(2-methoxy-phenylamino)-7-methyl-2-morpholin-4-yl-pyrido(1,2-a)pyrimidin-4-one;
7-methyl-2-morpholin-4-yl-9-o-tolylamino-pyrido(1,2-a)pyrimidin-4-one;
9-(3,4-dimethyl-phenylamino)-7-methyl-2-morpholin-4-yl-pyrido(1,2-a)pyrimidin-4-one;
7-methyl-9-(3-methyl-benzylamino)-2-morpholin-4-yl-pyrido(1,2-a)pyrimidin-4-one;
9-(2,3-dimethyl-phenylamino)-7-methyl-2-morpholin-4-yl-pyrido(1,2-a)pyrimidin-4-one;
7-methyl-9-(2-methyl-benzylamino)-2-morpholin-4-yl-pyrido(1,2-a)pyrimidin-4-one;
5-morpholin-4-yl-2-nitro-phenylamine; 1-(2-hydroxy-4-morpholin-4-yl-phenyl)-phenyl-methanone; and, 2-chloro-1-(2-hydroxy-4-morpholin-4-yl-phenyl)-ethanone.

Pharmaceutically acceptable salts” means any salts that are physiologically acceptable insofar as they are compatible with other ingredients of the formulation and not deleterious to the recipient thereof. Some specific preferred examples are: acetate, trifluoroacetate, hydrochloride, hydrobromide, sulfate, citrate, tartrate, glycolate, oxalate.

Administration of prodrugs is also contemplated. The term “prodrug” as used herein refers to compounds that are rapidly transformed in vivo to a more pharmacologically active compound. Prodrug design is discussed generally in Hardma, et al. (Eds.), Goodman and Gilman's The Pharmacological Basis of Therapeutics, 9th ed., pp. 11-16 (1996). A thorough discussion is provided in Higuchi, et al., Prodrugs as Novel Delivery Systems, Vol. 14, ASCD Symposium Series, and in Roche (ed.), Bioreversible Carriers in Drug Design, American Pharmaceutical Association and Pergamon Press (1987).

To illustrate, prodrugs can be converted into a pharmacologically active form through hydrolysis of, for example, an ester or amide linkage, thereby introducing or exposing a functional group on the resultant product. The prodrugs can be designed to react with an endogenous compound to form a water-soluble conjugate that further enhances the pharmacological properties of the compound, for example, increased circulatory half-life. Alternatively, prodrugs can be designed to undergo covalent modification on a functional group with, for example, glucuronic acid, sulfate, glutathione, amino acids, or acetate. The resulting conjugate can be inactivated and excreted in the urine, or rendered more potent than the parent compound. High molecular weight conjugates also can be excreted into the bile, subjected to enzymatic cleavage, and released back into the circulation, thereby effectively increasing the biological half-life of the originally administered compound.

Additionally, compounds that selectively negatively regulate p110δ mRNA expression more effectively than they do other isozymes of the PI3K family, and that possess acceptable pharmacological properties are contemplated for use as PI3Kδ selective inhibitors in the methods of the invention. Polynucleotides encoding human p110δ are disclosed, for example, in Genbank Accession Nos. AR255866, NM 005026, U86453, U57843 and Y10055, the entire disclosures of which are incorporated herein by reference [see also, Vanhaesebroeck, et al., Proc. Natl. Acad. Sci., 94:4330-4335 (1997), the entire disclosure of which is incorporated herein by reference]. Representative polynucleotides encoding mouse p110δ are disclosed, for example, in Genbank Accession Nos. BC035203, AK040867, U86587, and NM_{—}008840, and a polynucleotide encoding rat p110δ is disclosed in Genbank Accession No. XM_{—}345606, in each case the entire disclosures of which are incorporated herein by reference.

In one embodiment, the invention provides methods using antisense oligonucleotides which negatively regulate p110δ expression via hybridization to messenger RNA (mRNA) encoding p110δ. Suitable antisense oligonucleotide molecules are disclosed in U.S. Pat. No. 6,046,049, the entire disclosure of which is incorporated herein by reference. In one aspect, antisense oligonucleotides at least 5 to about 50 nucleotides in length, including all lengths (measured in number of nucleotides) in between, which specifically hybridize to mRNA encoding p110δ and inhibit mRNA expression, and as a result p110δ protein expression, are contemplated for use in the methods of the invention. Antisense oligonucleotides include those comprising modified internucleotide linkages and/or those comprising modified nucleotides which are known in the art to improve stability of the oligonucleotide, i.e., make the oligonucleotide more resistant to nuclease degradation, particularly in vivo. It is understood in the art that, while antisense oligonucleotides that are perfectly complementary to a region in the target polynucleotide possess the highest degree of specific inhibition, antisense oligonucleotides that are not perfectly complementary, i.e., those which include a limited number of mismatches with respect to a region in the target polynucleotide, also retain high degrees of hybridization specificity and therefore also can inhibit expression of the target mRNA. Accordingly, the invention contemplates methods using antisense oligonucleotides that are perfectly complementary to a target region in a polynucleotide encoding p110δ, as well as methods that utilize antisense oligonucleotides that are not perfectly complementary (i.e., include mismatches) to a target region in the target polynucleotide to the extent that the mismatches do not preclude specific hybridization to the target region in the target polynucleotide. Preparation and use of antisense compounds is described, for example, in U.S. Pat. No. 6,277,981, the entire disclosure of which is incorporated herein by reference [see also, Gibson (Ed.), Antisense and Ribozyme Methodolozy, (1997), the entire disclosure of which is incorporated herein by reference].

The invention further contemplates methods utilizing ribozyme inhibitors which, as is known in the art, include a nucleotide region which specifically hybridizes to a target polynucleotide and an enzymatic moiety that digests the target polynucleotide. Specificity of ribozyme inhibition is related to the length the antisense region and the degree of complementarity of the antisense region to the target region in the target polynucleotide. The methods of the invention therefore contemplate ribozyme inhibitors comprising antisense regions from 5 to about 50 nucleotides in length, including all nucleotide lengths in between, that are perfectly complementary, as well as antisense regions that include mismatches to the extent that the mismatches do not preclude specific hybridization to the target region in the target p110δ-encoding polynucleotide. Ribozymes useful in methods of the invention include those comprising modified internucleotide linkages and/or those comprising modified nucleotides which are known in the art to improve stability of the oligonucleotide, i.e., make the oligonucleotide more resistant to nuclease degradation, particularly in vivo, to the extent that the modifications do not alter the ability of the ribozyme to specifically hybridize to the target region or diminish enzymatic activity of the molecule. Because ribozymes are enzymatic, a single molecule is able to direct digestion of multiple target molecules thereby offering the advantage of being effective at lower concentrations than non-enzymatic antisense oligonucleotides. Preparation and use of ribozyme technology is described in U.S. Pat. Nos. 6,696,250, 6,410,224, 5,225,347, the entire disclosures of which are incorporated herein by reference.

The invention also contemplates use of methods in which RNAi technology is utilized for inhibiting p110δ expression. In one aspect, the invention provides double-stranded RNA (dsRNA) wherein one strand is complementary to a target region in a target p110δ-encoding polynucleotide. In general, dsRNA molecules of this type are less than 30 nucleotides in length and referred to in the art as short interfering RNA (siRNA). The invention also contemplates, however, use of dsRNA molecules longer than 30 nucleotides in length, and in certain aspects of the invention, these longer dsRNA molecules can be about 30 nucleotides in length up to 200 nucleotides in length and longer, and including all length dsRNA molecules in between. As with other RNA inhibitors, complementarity of one strand in the dsRNA molecule can be a perfect match with the target region in the target polynucleotide, or may include mismatches to the extent that the mismatches do not preclude specific hybridization to the target region in the target p110δ-encoding polynucleotide. As with other RNA inhibition technologies, dsRNA molecules include those comprising modified internucleotide linkages and/or those comprising modified nucleotides which are known in the art to improve stability of the oligonucleotide, i.e., make the oligonucleotide more resistant to nuclease degradation, particularly in vivo. Preparation and use of RNAi compounds is described in U.S. patent application Ser. No. 2004/0023390, the entire disclosure of which is incorporated herein by reference.

The invention further contemplates methods wherein inhibition of p110δ is effected using RNA lasso technology. Circular RNA lasso inhibitors are highly structured molecules that are inherently more resistant to degradation and therefore do not, in general, include or require modified internucleotide linkage or modified nucleotides. The circular lasso structure includes a region that is capable of hybridizing to a target region in a target polynucleotide, the hybridizing region in the lasso being of a length typical for other RNA inhibiting technologies. As with other RNA inhibiting technologies, the hybridizing region in the lasso may be a perfect match with the target region in the target polynucleotide, or may include mismatches to the extent that the mismatches do not preclude specific hybridization to the target region in the target p110δ-encoding polynucleotide. Because RNA lassos are circular and form tight topological linkage with the target region, inhibitors of this type are generally not displaced by helicase action unlike typical antisense oligonucleotides, and therefore can be utilized as dosages lower than typical antisense oligonucleotides. Preparation and use of RNA lassos is described in U.S. Pat. No. 6,369,038, the entire disclosure of which is incorporated herein by reference.

The inhibitors of the invention may be covalently or noncovalently associated with a carrier molecule including but not limited to a linear polymer (e.g., polyethylene glycol, polylysine, dextran, etc.), a branched-chain polymer (see U.S. Pat. Nos. 4,289,872 and 5,229,490; PCT Publication No. WO 93/21259), a lipid, a cholesterol group (such as a steroid), or a carbohydrate or oligosaccharide. Specific examples of carriers for use in the pharmaceutical compositions of the invention include carbohydrate-based polymers such as trehalose, mannitol, xylitol, sucrose, lactose, sorbitol, dextrans such as cyclodextran, cellulose, and cellulose derivatives. Also, the use of liposomes, microcapsules or microspheres, inclusion complexes, or other types of carriers is contemplated.

Other carriers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Still other useful carrier polymers known in the art include monomethoxy-polyethylene glycol, poly-(N-vinyl pyrrolidone)-polyethylene glycol, propylene glycol homopolymers, a polypropylene oxidelethylene oxide co-polymer, polyoxyethylated polyols (e.g., glycerol) and polyvinyl alcohol, as well as mixtures of these polymers.

Derivitization with bifunctional agents is useful for cross-linking a compound of the invention to a support matrix or to a carrier. One such carrier is polyethylene glycol (PEG). The PEG group may be of any convenient molecular weight and may be straight chain or branched. The average molecular weight of the PEG can range from about 2 kDa to about 100 kDa, in another aspect from about 5 kDa to about 50 kDa, and in a further aspect from about 5 kDa to about 10 kDa. The PEG groups will generally be attached to the compounds of the invention via acylation, reductive alkylation, Michael addition, thiol alkylation or other chemoselective conjugation/ligation methods through a reactive group on the PEG moiety (e.g., an aldehyde, amino, ester, thiol, ci-haloacetyl, maleimido or hydrazino group) to a reactive group on the target inhibitor compound (e.g., an aldehyde, amino, ester, thiol, a-haloacetyl, maleimido or hydrazino group). Cross-linking agents can include, e.g., esters with 4-azidosalicylic acid, homobifunctional imidoesters, including disuccinimidyl esters such as 3,3′-dithiobis (succinimidylpropionate), and bifunctional maleimides such as bis-N-maleimido-1,8-octane. Derivatizing agents such as methyl-3-[(p-azidophenyl)dithiolpropioimidate yield photoactivatable intermediates that are capable of forming crosslinks in the presence of light. Alternatively, reactive water-insoluble matrices such as cyanogen bromide-activated carbohydrates and the reactive substrates described in U.S. Pat. Nos. 3,969,287; 3,691,016; 4,195,128; 4,247,642; 4,229,537; and 4,330,440 may be employed for inhibitor immobilization.

The pharmaceutical compositions of the invention may also include compounds derivatized to include one or more antibody Fc regions. Fc regions of antibodies comprise monomeric polypeptides that may be in dimeric or multimeric forms linked by disulfide bonds or by non-covalent association. The number of intermolecular disulfide bonds between monomeric subunits of Fc molecules can be from one to four depending on the class (e.g., IgG, IgA, IgE) or subclass (e.g., IgG1, IgG2, IgG3, IgA1, IgGA2) of antibody from which the Fc region is derived. The term “Fc” as used herein is generic to the monomeric, dimeric, and multimeric forms of Fc molecules, with the Fc region being a wild type structure or a derivatized structure. The pharmaceutical compositions of the invention may also include the salvage receptor binding domain of an Fc molecule as described in WO 96/32478, as well as other Fc molecules described in WO 97/34631.

Such derivatized moieties preferably-improve one or more characteristics of the inhibitor compounds of the invention, including for example, biological activity, solubility, absorption, biological half life, and the like. Alternatively, derivatized moieties result in compounds that have the same, or essentially the same, characteristics and/or properties of the compound that is not derivatized. The moieties may alternatively eliminate or attenuate any undesirable side effect of the compounds and the like.

Methods include administration of an inhibitor by itself, or in combination as described herein, and in each case optionally including one or more suitable diluents, fillers, salts, disintegrants, binders, lubricants, glidants, wetting agents, controlled release matrices, colorants/flavoring, carriers, excipients, buffers, stabilizers, solubilizers, other materials well known in the art and combinations thereof.

Any pharmaceutically acceptable (i.e., sterile and non-toxic) liquid, semisolid, or solid diluents that serve as pharmaceutical vehicles, excipients, or media may be used. Exemplary diluents include, but are not limited to, polyoxyethylene sorbitan monolaurate, magnesium stearate, calcium phosphate, mineral oil, cocoa butter, and oil of theobroma, methyl- and propylhydroxybenzoate, talc, alginates, carbohydrates, especially mannitol, α-lactose, anhydrous lactose, cellulose, sucrose, dextrose, sorbitol, modified dextrans, gum acacia, and starch. Some commercially available diluents are Fast-Flo®, Emdex®, STA-Rx® 1500, Emcompress® and Avicel®. Such compositions may influence the physical state, stability, rate of in vivo release, and rate of in vivo clearance of the PI3Kδ inhibitor compounds [see, e.g., Remington's Pharmaceutical Sciences, 18th Ed. pp. 1435-1712 (1990), which is incorporated herein by reference].

Pharmaceutically acceptable fillers can include, for example, lactose, microcrystalline cellulose, dicalcium phosphate, tricalcium phosphate, calcium sulfate, dextrose, mannitol, and/or sucrose.

Inorganic salts including calcium triphosphate, magnesium carbonate, and sodium chloride may also be used as fillers in the pharmaceutical compositions. Amino acids may be used such as use in a buffer formulation of the pharmaceutical compositions.

Disintegrants may be included in solid dosage formulations of the inhibitors. Materials used as disintegrants include but are not limited to starch including the commercial disintegrant based on starch, Explotab®. Sodium starch glycolate, Amberlite, sodium carboxymethylcellulose, ultramylopectin, sodium alginate, gelatin, orange peel, acid carboxymethylcellulose, natural sponge and bentonite may all be used as disintegrants in the pharmaceutical compositions. Other disintegrants include insoluble cationic exchange resins. Powdered gums including powdered gums such as agar, Karaya or tragacanth may be used as disintegrants and as binders. Alginic acid and its sodium salt are also useful as disintegrants.

Binders may be used to hold the therapeutic agent together to form a hard tablet and include materials from natural products such as acacia, tragacanth, starch and gelatin. Others include methyl cellulose (MC), ethyl cellulose (EC) and carboxymethyl cellulose (CMC). Polyvinyl pyrrolidone (PVP) and hydroxypropylmethyl cellulose (HPMC) can both be used in alcoholic solutions to facilitate granulation of the therapeutic ingredient.

An antifrictional agent may be included in the formulation of the therapeutic ingredient to prevent sticking during the formulation process. Lubricants may be used as a layer between the therapeutic ingredient and the die wall, and these can include but are not limited to; stearic acid including its magnesium and calcium salts, polytetrafluoroethylene (PTFE), liquid paraffin, vegetable oils and waxes. Soluble lubricants may also be used such as sodium lauryl sulfate, magnesium lauryl sulfate, polyethylene glycol of various molecular weights, Carbowax® 4000 and 6000.

Glidants that might improve the flow properties of the therapeutic ingredient during formulation and to aid rearrangement during compression might be added. Suitable glidants include starch, talc, pyrogenic silica and hydrated silicoaluminate.

To aid dissolution of the therapeutic into the aqueous environment, a surfactant might be added as a wetting agent. Natural or synthetic surfactants may be used. Surfactants may include anionic detergents such as sodium lauryl sulfate, dioctyl sodium sulfosuccinate, and dioctyl sodium sulfonate. Cationic detergents such as benzalkonium chloride and benzethonium chloride may be used. Nonionic detergents that can be used in the pharmaceutical formulations include lauromacrogol 400, polyoxyl 40 stearate, polyoxyethylene hydrogenated castor oil 10, 50 and 60, glycerol monostearate, polysorbate 40, 60, 65 and 80, sucrose fatty acid ester, methyl cellulose and carboxymethyl cellulose. These surfactants can be present in the pharmaceutical compositions of the invention either alone or as a mixture in different ratios.

Controlled release formulation may be desirable. The inhibitors of the invention can be incorporated into an inert matrix which permits release by either diffusion or leaching mechanisms, e.g., gums. Slowly degenerating matrices may also be incorporated into the pharmaceutical formulations, e.g., alginates, polysaccharides. Another form of controlled release is a method based on the Oros® therapeutic system (Alza Corp.), i.e., the drug is enclosed in a semipermeable membrane which allows water to enter and push the inhibitor compound out through a single small opening due to osmotic effects. Some enteric coatings also have a delayed release effect.

Colorants and flavoring agents may also be included in the pharmaceutical compositions. For example, the inhibitors of the invention may be formulated (such as by liposome or microsphere encapsulation) and then further contained within an edible product, such as a beverage containing colorants and flavoring agents.

The therapeutic agent can also be given in a film coated tablet. Nonenteric materials for use in coating the pharmaceutical compositions include methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, povidone and polyethylene glycols. Enteric materials for use in coating the pharmaceutical compositions include esters of phthalic acid. A mix of materials might be used to provide the optimum film coating. Film coating manufacturing may be carried out in a pan coater, in a fluidized bed, or by compression coating.

The compositions can be administered in solid, semi-solid, liquid or gaseous form, or may be in dried powder, such as lyophilized form. The pharmaceutical compositions can be packaged in forms convenient for delivery, including, for example, capsules, sachets, cachets, gelatins, papers, tablets, capsules, suppositories, pellets, pills, troches, lozenges or other forms known in the art. The type of packaging will generally depend on the desired route of administration. Implantable sustained release formulations are also contemplated, as are transdermal formulations.

In the methods according to the invention, the inhibitor compounds may be administered by various routes. For example, pharmaceutical compositions may be for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded-under the-splenic capsule, brain, or in the cornea); by sublingual, anal, vaginal, or by surgical implantation, e.g., embedded under the splenic capsule, brain, or in the cornea. The treatment may consist of a single dose or a plurality of doses over a period of time. In general, the methods of the invention involve administering effective amounts of an inhibitor of the invention together with pharmaceutically acceptable diluents, preservatives, solubilizers, emulsifiers, adjuvants and/or carriers, as described above.

In one aspect, the invention provides methods for oral administration of a pharmaceutical composition of the invention. Oral solid dosage forms are described generally in Remington's Pharmaceutical Sciences, supra at Chapter 89. Solid dosage forms include tablets, capsules, pills, troches or lozenges, and cachets or pellets. Also, liposomal or proteinoid encapsulation may be used to formulate the compositions (as, for example, proteinoid microspheres reported in U.S. Pat. No. 4,925,673). Liposomal encapsulation may include liposomes that are derivatized with various polymers (e.g., U.S. Pat. No. 5,013,556). In general, the formulation will include a compound of the invention and inert ingredients which protect against degradation in the stomach and which permit release of the biologically active material in the intestine.

The inhibitors can be included in the formulation as fine multiparticulates in the form of granules or pellets of particle size about I mm. The formulation of the material for capsule administration could also be as a powder, lightly compressed plugs or even as tablets. The capsules could be prepared by compression.

Also contemplated herein is pulmonary delivery of the PI3Kδ inhibitors in accordance with the invention. According to this aspect of the invention, the inhibitor is delivered to the lungs of a mammal while inhaling and traverses across the lung epithelial lining to the blood stream.

Contemplated for use in the practice of this invention are a wide range of mechanical devices designed for pulmonary delivery of therapeutic products, including but not limited to nebulizers, metered dose inhalers, and powder inhalers, all of which are familiar to those skilled in the art. Some specific examples of commercially available devices suitable for the practice of this invention are the UltraVent® nebulizer, manufactured by Mallinckrodt, Inc., St. Louis, Mo.; the Acorn II® nebulizer, manufactured by Marquest Medical Products, Englewood, Colo.; the Ventolin® metered dose inhaler, manufactured by Glaxo Inc., Research Triangle Park, N.C.; and the Spinhaler® powder inhaler, manufactured by Fisons Corp., Bedford, Mass.

All such devices require the use of formulations suitable for the dispensing of the inventive compound. Typically, each formulation is specific to the type of device employed and may involve the use of an appropriate propellant material, in addition to diluents, adjuvants and/or carriers useful in therapy.

When used in pulmonary administration methods, the inhibitors of the invention are most advantageously prepared in particulate form with an average particle size of less than 10 μm (or microns), for example, 0.5 to 5 μm, for most effective delivery to the distal lung.

Formulations suitable for use with a nebulizer, either jet or ultrasonic, will typically comprise the inventive compound dissolved in water at a concentration range of about 0.1 to 100 mg of inhibitor per mL of solution, 1 to 50 mg of inhibitor per mL of solution, or 5 to 25 mg of inhibitor per mL of solution. The formulation may also include a buffer. The nebulizer formulation may also contain a surfactant, to reduce or prevent surface induced aggregation of the inhibitor caused by atomization of the solution in forming the aerosol.

Formulations for use with a metered-dose inhaler device will generally comprise a finely divided powder-containing the inventive inhibitors suspended in a propellant with the aid of a surfactant. The propellant may be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid may also be useful as a surfactant.

Formulations for dispensing from a powder inhaler device will comprise a finely divided dry powder containing the inventive compound and may also include a bulking agent or diluent such as lactose, sorbitol, sucrose, mannitol, trehalose, or xylitol in amounts which facilitate dispersal of the powder from the device,. e.g., 50 to 90% by weight of the formulation.

Nasal delivery of the inventive compound is also contemplated. Nasal delivery allows the passage of the inhibitor to the blood stream directly after administering the therapeutic product to the nose, without the necessity for deposition of the product in the lung. Formulations for nasal delivery may include dextran or cyclodextran. Delivery via transport across other mucous membranes is also contemplated.

Toxicity and therapeutic efficacy of the PI3Kδ selective compounds can be determined by standard pharmaceutical procedures in cell cultures or experimental animals, e.g., for determining the LD50 (the dose lethal to 50% of the population) and the ED50 (the dose therapeutically effective in 50% of the population). Additionally, this information can be determined in cell cultures or experimental animals additionally treated with other therapies including but not limited to radiation, chemotherapeutic agents, photodynamic therapies, radiofrequency ablation, anti-angiogenic agents, and combinations thereof.

In practice of the methods of the invention, the pharmaceutical compositions are generally provided in doses ranging from 1 pg compound/kg body weight to 1000 mg/kg, 0.1 mg/kg to 100 mg/kg, 0.1 mg/kg to 50 mg/kg, and 1 to 20 mg/kg, given in daily doses or in equivalent doses at longer or shorter intervals, e.g., every other day, twice weekly, weekly, or twice or three times daily. The inhibitor compositions may be administered by an initial bolus followed by a continuous infusion to maintain therapeutic circulating levels of drug product. Those of ordinary skill in the art will readily optimize effective dosages and administration regimens as determined by good medical practice and the clinical condition of the individual to be treated. The frequency of dosing will depend on the pharmacokinetic parameters of the agents and the route of administration. The optimal pharmaceutical formulation will be determined by one skilled in the art depending upon the route of administration and desired dosage [see, for example, Remington's Pharmaceutical Sciences, pp. 1435-1712, the disclosure of which is hereby incorporated by reference]. Such formulations may influence the physical state, stability, rate of in vivo release, and rate of in vivo clearance of the administered agents. Depending on the route of administration, a suitable dose may be calculated according to body weight, body surface area or organ size. Further refinement of the calculations necessary to determine the appropriate dosage for treatment involving each of the above mentioned formulations is routinely made by those of ordinary skill in the art without undue experimentation, especially in light of the dosage information and assays disclosed herein, as well as the pharmacokinetic data observed in human clinical trials. Appropriate dosages may be ascertained by using established assays for determining blood level dosages in conjunction with an appropriate physician considering various factors which modify the action of drugs, e.g., the drug's specific activity, the severity of the indication, and the responsiveness of the individual, the age, condition, body weight, sex and diet of the individual, the time of administration and other clinical factors. As studies are conducted, further information will emerge regarding the appropriate dosage levels and duration of treatment for various diseases and conditions capable of being treated with the methods of the invention.

### Examples

The following examples are provided to illustrate the—invention, but are not intended to limit the scope thereof. Example 1 provides some of the reagents used in Examples 2-5. Examples 2-5 provide in vivo and in vitro evidence that PI3Kδ selective inhibitors inhibit immune responses stimulated by endogenous factors without substantially inhibiting immune responses stimulated by exogenous factors and/or immune responsiveness.

### Example 1

### Reagents

Monoclonal antibodies (mAb) and cell lines used in experiments included the ICAM-1 mAb RR 1/1 (Biosource International, Camarillo, Calif.), FITC-conjugated goat F(ab′)₂ anti-mouse Ig (CALTAG Laboratories, Burlingame, Calif.), E-selectin mAb CL3 (ATCC, Manassas, Va.), FITC-conjugated Gr-1 (BD Pharmingen, Franklin Lakes, N.J.), anti-Akt and PI3Kδ (Santa Cruz, Calif.), horseradish peroxidase-conjugated secondary antibodies (Jackson ImmunoResearch Laboratories Inc., West Grove, Pa.), CHO-ICAM-1 cells (ATCC, Manassas, Va.). Inflammatory agents and chemoattractants used included murine recombinant TNFα (PeproTech, Inc., Rocky Hill, N.J.), human recombinant TNFα (R&D Systems, Minneapolis, Minn.), LTB₄ (BIOMOL, Plymouth Meeting, Pa.), fMLP (Sigma, St. Louis, Mo.), C5a (Sigma) and IL-8 (R&D Systems). A small molecule selective PI3Kδ inhibitor in accordance with the invention was synthesized and purified as described by Sadhu, et al., J. Immunol., 170:2647-2654 (2003).

### Example 2

### PI3Kδ Inhibitor Selectivity

The selectivity of an inhibitor in accordance with the invention (10 μM) was tested against several human protein kinases and a phosphatase. Protein kinase assays were performed in the presence of 100 μM ATP. The kinase activities marked with an asterisk were reported by Sadhu, et al., J. Immunol., 170:2647-2654 (2003).

TABLE 1
PI3Kδ selective inhibitor effect on the activity of various enzymes.

| Enzyme | Activity (% of control) ± SD |
|---|---|
| EGF receptor tyrosine kinase | 102 ± 5.5 |
| Insulin receptor tyrosine kinase | 98 ± 6.2 |
| CD45 tyrosine phosphatase | 104 ± 2.2 |
| PKC-θ | 97 ± 5.5 |
| PDK1 | 91.5 ± 2.1 |
| Lck | 116.5 ± 9.2 |
| P70S6K | 98.5 ± 0.7 |
| CDK2/cyclinA | 92.5 ± 2.12 |
| ZAP-70 | 97.5 ± 13.4 |
| p38 MAPK | No inhibition* |
| DNA-PK | No inhibition* |
| CHK1 | No inhibition* |
| cSrc | No inhibition* |
| CK1 | No inhibition* |
| PKBα (Akt 1) | No inhibition* |
| PKCα | No inhibition* |
| PKCβII | No inhibition* |

### Example 3

### PI3Kδ Catalytic Activity is Preferentially Utilized by Different Chemoattractant Receptors and Their Ligands

It is known that distinct signal transduction pathways are utilized by host-derived versus bacteria-produced chemoattractants [Heit, J. Cell Biol., 159:91-102 (2002)]. To determine whether specific chemotactic agents preferentially rely on PI3Kδ in order to promote directed cell migration, the effect of inhibiting PI3Kδ on the ability of neutrophils to undergo chemotaxis was examined using a Transwell® assay system.

Neutrophil chemotaxis experiments were conducted as described [Roth, et al., J. Immunol. Methods, 188:97-116 (1995)]. Briefly, purified human neutrophils were incubated with DMSO (0.3% v/v) or an inhibitor in accordance with the invention reconstituted in DMSO (0.3%) for 20 minutes at room temperature. Cells were added to bare fiIter inserts (Transwell® 5 μm pore size; Coming Costar, Cambridge, Mass.), that were placed into wells containing chemoattractants or control medium of a Ultra low 24-well cluster plate, and incubated for 1 hour at 37° C. in a 5% CO₂ humidified environment. The number of neutrophils that migrated into the bottom well was determined by FACScan™ (Becton Dickinson, San Jose, Calif.). Results were expressed as percent neutrophil migration relative to the control (medium without inhibitor).

Dose response curves were generated to determine the concentrations of each chemoattractant, both host and bacterial-derived, necessary to support half-maximal migration. These values were 0.25 nM, 0.35 nM, 0.37 nM, and 1.25 nM for LTB₄, IL-8, C5a, and fMLP, respectively, and are in close agreement with previously reported results [Psychoyos, et al., J. Immunol. Methods, 137:37-46 (1991)].

PI3Kδ inhibition with an inhibitor according to the invention more potently diminished neutrophil migration in response to IL-8 and LTB₄ than fMLP. More specifically, a 10 to 17-fold lower concentration of inhibitor was required to achieve a 50 percent reduction (EC₅₀) in neutrophil chemotaxis to these host-derived chemoattractants as compared to the bacterial product, fMLP (0.61 μM and 1.1 μM versus 10.25 μM, respectively). Additionally, directed neutrophil migration was reduced by about 60% in the presence of IL-8 or LTB₄ versus about 30% in response to fMLP at a concentration of inhibitor (2.2 μM) that significantly inhibits PI3Kδ (>90%) but hot PI3Kα, β or γ activity.

Other PI3Kδ inhibitors in accordance with the invention also preferentially inhibited neutrophil migration towards LTB₄ than fMLP (EC₅₀ values ˜0.1 μM versus >10 μM, respectively). These data suggest that PI3Kδ is preferentially involved in neutrophil migration towards host-derived chemoattractants.

Accordingly, the Akt-phosphorylation signal transduction pathway in neutrophils appears to be utilized preferentially by host-derived chemoattractants as inhibition of PI3Kδ activity had a more pronounced effect on directed neutrophil migration and activation in response to endogenous factors such as LTB₄ and IL-8 than exogenous factors such as fMLP. Thus, the inhibition of PI3Kδ activity may provide a therapeutic benefit in specific inflammatory conditions as its activity is required for neutrophil migration to selective chemoattractants.

### Example 4

### The Preferential Role of PI3Kδ in Neutrophil Activation by Host-Derived Agonists

It has been suggested that class I PI3Ks are involved in neutrophil activation. For example, LY294002 inhibits all class la PI3Ks and other protein kinases, and has been shown to reduce fMLP-stimulated superoxide generation in these cells [Davies, et al., Biochem. J., 351:95-105 (2000); and, Vlahos, et al., J. Immunol., 154:2413-2422 (1995)].

To determine whether PI3Kδ contributes to agonist-induced activation, the ability of an inhibitor in accordance with the invention to selectively inhibit LTB₄ relative to fMLP-induced respiratory burst was evaluated in neutrophils in accordance with the following protocol.

Superoxide production by activated neutrophils was quantified spectrophotometrically [Tan, et al., J. Immunol. Meths., 238:59-68 (2000)]. Purified cells (1=10⁵ per ml)-were resuspended in HBSS containing 500 μM of 2-(4-lodophenyl)-3-(4-nitrophenyl)-5-(2,4-disulfo-phenyl)-2H-tetrazolium, monosodium salt (WST-1) (Dojindo Molecular Technologies, Inc., Gaithersburg, Md.), and 20 μg/ml human catalase (Sigma). An inhibitor in accordance with the invention or LY294002 (a non-selective PI3K inhibitor) in DMSO or DMSO alone was then added (10 minutes, RT) and the reaction initiated by adding 100 nM FMLP or 2 nM LTB₄. Absorbance at 450 nm was measured after an incubation period of 1 hour (SpectraMax®; Molecular Devices Corporation, Sunnyvale Calif.). The background WST-1 reduction in the absence of neutrophil stimulation was determined by incubation with 20 μg/ml superoxide dismutase (Roche Applied Science, Indianapolis, Ind.). Results were expressed as the percentage of superoxide produced relative to the control (medium without inhibitor).

At concentrations of fMLP (100 nM) or LTB₄ (2 nM) that represent half-maximal production of superoxide anions, an inhibitor in accordance with the invention more potently reduced the response to LTB₄ than to fMLP. For instance, a 13-fold lower concentration of an inhibitor in accordance with the invention was required to achieve a 50% reduction in superoxide anion production in response to LTB₄ versus the bacterial agonist, fMLP.

In addition to an agonist-stimulated respiratory burst, activated neutrophils also released the contents of their granules that include proteases such as elastase [Borregaard, et al., Blood, 89:3503-3521 (1997)]. Neutrophil elastase release assays were performed in accordance with the following protocol.

Microtiter assays for the detection of elastase released from purified neutrophils (1.1×10⁵ per well in PBS) were performed in the absence or presence of an inhibitor in accordance with the invention or LY294002 on fibrinogen-coated plates [Mulligan, et al., Proc. Natl. Acad. Sci. U.S.A., 90:11523-11527 (1993)]. Neutrophils were stimulated with exocytosis buffer (endotoxin free water containing 10 μg/mi cytochalasin B, 500 μg/ml L-methionine and either 20 nM FMLP, 2 nM LTB₄ or 1 nM TNFα) for 60 minutes at 37° C. The samples were centrifuged and 90 μl of the supernatant was transferred to another plate containing 10 μl of methoxysuccinyl-alanylalanylproly- lvalyl-p-nitroanilide (10 mM, Sigma). Absorbance at 410 nm was measured at one hour as described above. Results are expressed as the percentage elastase activity relative to the control (medium without inhibitor).

To determine the ability of inhibitors in accordance with the invention to impede agonist-induced neutrophil degranulation, the effect of an inhibitor on elastase exocytosis in response to 20 nM fMLP or 2 nM LTB₄ (concentrations which represent half-maximal release of this protease) were measured.

A role for PI3Kδ in this process is suggested by the ability of a compound in accordance with the invention to impair elastase exocytosis from neutrophils in a dose dependent manner. Importantly, a concentration of this inhibitor which was approximately 50-fold less was required to achieve a half-maximal release of this protease in response to LTB₄ versus fMLP (0.03 μM versus 1.67 μM, respectively). Furthermore, a compound in accordance with the invention also reduced TNFα-mediated degranulation of neutrophils by more than 90% at a concentration that primarily impacts on the biochemical activity of PI3Kδ (5 μM). TNFα production in leukocytes in response to LPS, however, was not significantly impaired at this concentration of inhibitor.

This conclusion that the PI3Kδ/Akt signal pathway is preferentially used by host agonists is also supported by the ability of inhibitor to impede neutrophil respiratory burst and degranulation in response to LTB₄ versus fMLP, processes that have not been previously shown to rely on this signal transduction pathway. These results suggest the possibility that pharmacological blockade of PI3Kδ activity may not significantly impair the ability of neutrophils to respond to bacterial pathogens.

### Example 5

### PI3Kδ Activity is Not Required for Host Clearance of Microbial Infection

Consistent with this hypothesis is the observation that PI3Kδ inhibition did not prevent host clearance in a systemic bacterial infection model.

Two separate bacterial clearance studies were conducted. The bacterial clearance studies measured the clearance of systemic Listeria monocytogenes organisms (˜10⁵ colony forming units (CFU) per rat, IV given at time=0) at 72 hours post-infection in Lewis rats (n=10 per treatment group) as determined by bacterial colony counts per gram of spleen tissue. Colony counts (CFU) per gram of spleen tissues were determined at necropsy, 72 hours post-infection and expressed as the log of the colony count (e.g., 100,000 CFU═log 5) per gram of tissue. Colony count measurements provide an indication of the extent of phagocytosis of bacteria by splenic neutrophils and macrophages.

The first study looked at clearance in groups treated with vehicle alone (PEG400), a compound in accordance with the invention (10 mg/kg, BID, PO), the same compound at an increased dose (50 mg/kg, BID, PO), and dexamethasone (2 mg/kg, BID, PO). The compound in accordance with the invention was a PI3Kδ selective inhibitor (IC50 for PI3Kδ=0.02 uM; IC50 for PI3Kα, PI3Kβ, and PI3Kγ=33, 5.2, 1.6 uM, respectively). A second, similar study was conducted with the same control treatment groups, but a different PI3Kδ selective inhibitor, which was somewhat less selective for PI3Kδ was used (IC50 for PI3Kδ=0.016 uM; IC50 for PI3Kα, PI3Kβ, and PI3Kγ=0.96, 0.22, 0.125 uM, respectively).

Results (shown in Table 2) should be compared only within study groups since the colony burdens produced by the inocula used in the two studies differ substantially. Nonetheless, the results show that the PI3Kδ selective inhibitors have undetectable or minimal effect, depending on dosage, on microbial clearance in spleen tissues compared to dexamethasone, a systemic corticosteroid similar to those prescribed for immune renal diseases and other auioimmune diseases such as lupus nephritis and rheumatoid arthritis. These experiments indicate that systemic bacterial clearance will remain effective and that PI3Kδ selective inhibitors in accordance with the invention are not as broadly immunosuppressive as the FDA-approved corticosteroid, dexamethasone, at an efficacious dose. Accordingly, administration of a compound in accordance with the invention does not substantially inhibit immune responsiveness.

The results further indicate that the first compound at the lower dose (10 mg/kg), spared the splenic neutrophil response such that systemic Listeria infections were cleared as effectively as infections in the vehicle control group and more effectively than those animals treated with dexamethasone (2 mg/kg). A similar dose of the same compound (8 mg/kg) was shown to be effective in reducing an antibody response to, sheep erythrocytes in rats by 61% and in reducing LPS-triggered neutrophil influx into the airway by 47%. Even the high dose (50 mg/kg) treatment group showed minimal inhibition of bacterial clearance and markedly less than was observed with the dexamethasone-treated group. Again, the results demonstrate that, administration of a compound in accordance with the invention does not substantially inhibit immune responsiveness.

Animals treated with a 10 mg/kg dose of the less selective compound showed somewhat higher colony counts than was seen in the vehicle control group. The increase in colony counts observed with either the 10 or 50 mg/kg dose groups of the less selective compound represented bacterial loads that were not lethal at 72 hours. On the other hand lethality was observed with the dexamethasone treatment group in the second experiment.

TABLE 2
The effect of an inhibitor in accordance with the
invention on the ability of host animals to clear systemic
Listeria monocytogenes infections.

| | Clearance | Clearance |
|---|---|---|
| | Experiment 1 | Experiment 2 |
| PI3K delta potency (uM) | 0.015 | 0.016 |
| PI3K alpha, beta, gamma | 33, 5.2, 1.6 | 0.96, 0.22, 0.125 |
| potency (uM) | | |
| Log colony count/gm spleen | 3.9 ± 0.25 | 6.8 ± 0.55 |
| tissue in vehicle group | | |
| (PEG400) | | |
| Log colony count/gm spleen | 4.06 ± 0.29 * | 8.61 ± 0.77 ** |
| tissue in 10 mg/kg dose | | |
| group | | |
| Log colony count/gm spleen | 4.38 ± 0.19 ** | 10.48 ± 1.52 ** |
| tissue in 50 mg/kg dose | | |
| group | | |
| Log colony count/gm spleen | Too numerous | Moribund at |
| tissue in dexamethasone | to count, >5 | 72 hours, >10 |
| group (2 mg/kg) | | |

* No statistically significant difference between vehicle and 10 mg/kg treatment group by one way ANOVA test
** P < 0.05, one way ANOVA test

Numerous modifications and variations in the invention as set forth in the above illustrative examples are expected to occur to those skilled in the art. Consequently only such limitations as appear in the appended claims should be placed on the invention.

1. A compound having formula (III) 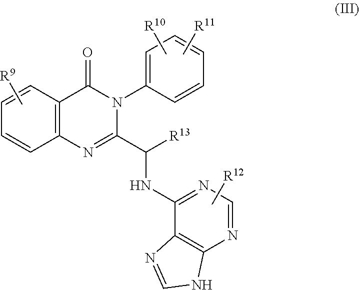
wherein R⁹, R¹⁰, R¹¹, and R¹², independently, are selected from the group consisting of hydrogen, C₁₋₆alkyl, or halo; and
R¹³ is selected from the group consisting of C₁₋₆alkyl;
or a pharmaceutically acceptable salt thereof.

2. The compound according to claim 1, wherein the compound is the S-enantiomer.

3. A pharmaceutical composition comprising an effective amount of a compound according to claim 1; and at least one pharmaceutically acceptable excipient.

4. A pharmaceutical composition comprising an effective amount of a compound according to claim 2 and at least one pharmaceutically acceptable excipient.

5. A method of treating a condition in an individual in need thereof, comprising administering to the individual a compound according to claim 1, wherein the condition is selected from the group consisting of asthma, allergic rhinitis, multiple sclerosis, diabetes, chronic obstructive pulmonary disease, cancer, and lymphoma.

6. A method of treating a condition in an individual in need thereof, comprising administering to the individual a compound according to claim 2, wherein the condition is selected from the group consisting of asthma, allergic rhinitis, multiple sclerosis, diabetes, chronic obstructive pulmonary disease, cancer, and lymphoma.

7. A method of treating a condition in an individual in need thereof, comprising administering to the individual a compound according to claim 3, wherein the condition is selected from the group consisting of asthma, allergic rhinitis, multiple sclerosis, diabetes, chronic obstructive pulmonary disease, cancer, and lymphoma.

8. A method of treating a condition in an individual in need thereof, comprising administering to the individual a compound according to claim 4, wherein the condition is selected from the group consisting of asthma, allergic rhinitis, multiple sclerosis, diabetes, chronic obstructive pulmonary disease, cancer, and lymphoma.
